(19) United States
(12) Patent Application Publication
(10) Pub. No.: US 2017/0309152 A1
(43) Pub. Date: Oct. 26, 2017

(54) SMART SAFETY APPARATUS, SYSTEM AND METHOD

(71) Applicant: Ulysses C. Dinkins, Marshall, WI (US)

(72) Inventor: Ulysses C. Dinkins, Marshall, WI (US)

(21) Appl. No.: 15/493,005

(22) Filed: Apr. 20, 2017

Related U.S. Application Data
(60) Provisional application No. 62/325,156, filed on Apr. 20, 2016.
(60) Provisional application No. 62/404,976, filed on Oct. 6, 2016.

(51) Int. Cl. G08B 21/04; H04L 29/08; A62B 35/00; A42B 3/30; A41D 19/00; A42B 3/14; A42B 3/04; H04W 76/02; A61F 11/08

(52) U.S. Cl. CPC G08B 21/0461; H04W 76/02; H04L 67/10; A62B 35/0025; A42B 3/303; A61F 11/08; A41D 19/0027; A42B 3/14; A42B 3/046; A42B 3/0446; A42B 3/042; A62B 35/0018

(57) ABSTRACT

The disclosed device, system, and method may comprise one or more devices for digital personal protective equipment. These devices may be network enabled wearables. A plurality of sensors, data transmission mechanisms, self-illuminating LEDs, and communication mechanisms may be embedded in a suspension or harness in various embodiments. A number of sensors may likewise be provided in a glove or earbud (ear plug). The device may measure various aspects of a wearer's movement and transmit that data over a data connection (such as a wifi or mobile connection) to a server. The server may house project and human resources data, worker and condition information, and materials and supplies data, according to various examples of embodiments. The system may further comprise one or more applications which may interpret the data provided.

### CROSS-REFERENCE TO RELATED APPLICATIONS

This application claims the benefit of U.S. Provisional Patent Application, Ser. No. 62/325,156 filed Apr. 20, 2016, entitled Apparatus and Method for Monitoring Job Site, and claims the benefit of U.S. Provisional Patent Application Ser. No. 62/404,976 filed Oct. 6, 2016, entitled UC Hardchip, the entire contents of each of which is hereby incorporated by reference herein in its entirety.

### FIELD

This application relates to the field of wearable safety devices and resource management.

### BACKGROUND

Monitoring resources, workers, and progress are key to enabling safe and timely construction projects. Large jobsites may employ hundreds of workers and millions of dollars' worth of supplies and equipment. Construction may be performed on a variety of aspects of the project simultaneously. Differing roles in the construction project may entail varying risks of bodily harm. Therefore, failure to coordinate, including monitoring workers, providing timely updates on progress, and keeping an accurate count of resources may result in loss of time, money, and risk injury.

Unfortunately, providing such coordination or oversight can be very difficult. For example, a problem may arise during the construction of a building. This problem may be noticed by an individual worker, who may notify a supervisor, who may notify a foreman, etc. until the appropriate party in the chain of command is located. Known systems may require a series of phone calls, attempts to describe physical conditions to those with decision-making powers, and scrambles to find appropriate personnel. Such systems consume significant resources, time and energy.

The disadvantages of such a system are especially apparent from a safety perspective. Injured workers may not have a means to promptly report a situation that could cause or is causing injury.

Various solutions have been proposed to address the problem of construction site monitoring—i.e. how to obtain worker status, progress, and resources information quickly and easily. Each of these solutions have various drawbacks.

For example, while construction monitoring systems have been proposed, these systems do not monitor a worker's individual conditions. In addition, known construction monitoring systems may require carrying additional items, such as a phone, PDA, smart safety glasses, or other specialized device.

Known construction monitoring systems therefore may not address health and safety issues, data management issues, or provide means of predicting (and thereby preventing) issues in the future.

### SUMMARY

Accordingly, a device, system, and method that overcomes the above deficiencies is disclosed. Various further advantages should be understood to one of ordinary skill in the art.

The device may comprise a digital personal protective equipment/“internet of things” wearable. A plurality of sensors, data transmission mechanisms, self-illuminating LEDs, and communication mechanisms may be embedded in a suspension safety harness or body harness (e.g. a full-body safety harness) in various embodiments. A number of sensors may likewise be provided in a glove or earbud. The device may measure various aspects of a wearer's movement and transmit that data over a data connection (such as a wifi or mobile connection) to a server. The server may house project and human resources data, worker and condition information, and materials and supplies data, according to various examples of embodiments. The system may further comprise one or more applications which may interpret the data provided.

The device, system, and method herein may advantageously allow for real-time conditions and locations of an individual such as construction worker to be ascertained. In addition, the device, system, and method herein may advantageously allow for real-time tracking and location of resources. The device, system, and method herein may allow for streamlining human resources functionality onsite and off-site. The device, system, and method herein may allow for enhanced project bidding functionality as better data is ascertained and stored.

This device may comprise a “smart” safety harness suspension which may be provided into a protective helmet or similar headgear. In various embodiments, the safety harness may be designed to be provided in a hardhat. In addition, the device may comprise a full body harness, earbud(s), and/or glove(s) having sensors. The devices may comprise an enterprise internet of things (IoT) wearable device(s) created for the measurement of the wearer's movement and immediate environment (atmospheric conditions). The device may allow the accurate measurement, recording and transmittal of data sets created from the active movement of the wearer. The device may allow for consistent caching, transmitting, and updating of a cloud server or back-end database/software with events/measurements made in relation to the active wearer's movement and other conditions. The data packages created using this system may be made available in real-time for use through a cloud/SAT database/software application user interface dashboard, or other software applications. The system may include data analytics. The data analytics may be facilitated, for example, by the cloud-based storage of data (data sets) provided from various sources including by the devices. A variety of software encompassing use of this data and analytics thereof may be understood as within the scope of this disclosure.

The database may be understood to be, in various embodiments, part of a cloud rendering service or cloud reporting system. Various data as described herein may be understood to be provided in this system. The advantages of this system and the data collected in the cloud system may be further understood from the disclosure herein.

The data sets (data packages) gathered from these devices may improve workplace and/or (but not limited to) extracurricular activity productivity and communications including rock-climbing, and rafting. The devices and system and method herein may improve the wearers (and jobsite) safety by accurate gauging and measuring of a wearer's bio-markers. The device(s) may include a Micro-Electrical-Mechanical-System (MEMS) and/or Nano-Electrical-Mechanical-System (EMS), certain sensors, a data transmission unit, a communication unit, and lights in various embodiments. The digital safety-harness/suspension may have a GPS antenna(s), MEMS/NEMS, and battery and/or conductive metals for electrical magnetic inductive charging in various embodiments, provided embedded in and/or stacked through-out the safety-harness/suspension or full body harness. The invention may also include a camera provided within the device. The camera may be a digital camera. The digital camera may be suitable for streaming video. The device may include temple bone-conductions speakers and bi-directional (or omnidirectional) microphone. The device may be suitable for human use—i.e. safe to the employees or workers wearing the device.

The various embodiments disclosed herein may beneficially allow for the non-invasive ability to accurately measure, cache, and transmit data sets created from the active movement of the wearer and atmospheric conditions. These data sets may be used to improve workplace and extracurricular activity production, and communications across a range of applications, including but not limited to rock-climbing, construction, horse jockeys, and rafting. The disclosed device, system, and method may improve wearer safety by applications which analyze and use the provided real-time updates. The software may include a variety of applications including a web-based platform for accurate record keeping, charting, and gauging/measuring including but not limited to the wearer's individual personal bio-markers.

The device, system, and method herein may include a head-protection safety apparatus comprising a suspension, a plurality of sensors provided within the suspension, a wireless data communication device, a wireless audio communication device, and a self-illuminating light-emitting diode. The device, system, and method herein may include a method for communicating information relating to a construction site, the method comprising: establishing a radio transceiver connection, obtaining a plurality of sensor data from a sensor unit provided on a suspension, transmitting the sensor data to a server over a wireless network, storing the sensor data in a database, and accessing the sensor data through a software. The device, system, and method herein may further comprise a charging device comprising: a linear rack having a plurality of hooks in communication with a charging coil.

The device, system, and method herein may further comprise a safety monitoring system comprising a suspension having one or more sensors, one or more wireless communication devices, and one or more self-illuminating light-emitting diodes; one or more identification tags; a server; and a local area network in communication with the suspension and server. The safety monitoring system may further comprise a body harness having one or more sensors in communication with the suspension, one or more gloves having one or more sensors in communication with the suspension, one or more earbuds having one or more sensors in communication with the suspension, a human resources portal, and a sustainable crew generator software.

### BRIEF DESCRIPTION OF DRAWINGS

Various examples of embodiments of the systems, devices, and methods according to this invention will be described in detail, with reference to the following figures, wherein:

FIG. 1 is a drawing of a suspension according to various embodiments.

FIG. 2a is a drawing of a side perspective of a helmet having a suspension, according to various embodiments.

FIG. 2b is a drawing of a rear perspective of a helmet having a suspension, according to various embodiments.

FIG. 3 is a drawing of an individual wearing a helmet having a suspension, according to various embodiments.

FIG. 4 is a drawing of a suspension having a flattened perspective, according to various embodiments.

FIG. 5a shows a worker having a helmet covering a suspension according to various embodiments.

FIG. 5b shows a second view of a worker having a protective helmet on covering a suspension according to various embodiments.

FIG. 6a shows a harness, according to various embodiments.

FIG. 6b shows a harness having sensors, according to various embodiments.

FIG. 6c shows a second harness having sensors, according to various embodiments.

FIG. 7 shows a second drawing of a harness, according to various embodiments.

FIG. 8a shows a drawing of a glove, according to various examples of embodiments.

FIG. 8b shows a second drawing of a glove, according to various examples of embodiments.

FIG. 9a shows a number of earbuds, according to various examples of embodiments.

FIG. 9b shows another embodiment of earbuds according to various examples of embodiments.

FIG. 10 shows an interaction of a number of system components, according to various embodiments.

FIG. 11a shows a system for performing the workflows for the system and method herein, according to various examples of embodiments.

FIG. 11b shows a second version of a system for performing the workflows for the system and method herein, according to various examples of embodiments.

FIG. 12 shows a first workflow of the system and method herein, according to various embodiments.

FIG. 13 shows a second workflow of the system and method herein, according to various embodiments.

FIG. 14 shows a third workflow of the system and method herein, according to various embodiments.

FIG. 15 shows a fourth workflow of the system and method herein, according to various embodiments.

FIG. 16 shows a fifth workflow of the system and method herein, according to various embodiments.

FIG. 17 shows a sixth workflow of the system and method herein, according to various embodiments. [EL1]

FIG. 18 shows a software workflow for use with the system and method herein, according to various embodiments.

FIG. 19a shows a first device charging station according to various examples of embodiments.

FIG. 19b shows a second device charging station according to various examples of embodiments.

FIG. 19c shows a clip for use with a device charging station, according to various examples of embodiments.

It should be understood that the drawings are not necessarily to scale. In certain instances, details that are not necessary to the understanding of the invention or render other details difficult to perceive may have been omitted. It should be understood, of course, that the invention is not necessarily limited to the particular embodiments illustrated herein.

### DETAILED DESCRIPTION

Referring to the Figures, a number of embodiments of systems and devices for monitoring a construction site are provided.

The system and method herein may include the use of one or more devices, applications, computers (including embedded and cloud storage), in various embodiments. The devices may include but are not limited to one or more of the following: suspension 100, harness 200, glove 300, earbud 400. A network and storage system 500, as well as a charging device 700 may also be provided

Referring more specifically to the suspension and harness, the suspension may be understood to encompass a safety harness suspension for protection of a head. In other words, the term “suspension” herein may be understood to comprise a device which is provided on a wearer's head for flexible protection against falls or head injuries. The suspension may be provided into a suitable shell, such as a helmet.

In contrast, the term “harness” may be understood to encompass a safety harness for protection against falling, for example, from a height. The harness may be a full-body safety harness as described further herein. The harness may be used in connection with a lanyard, yo-yo, or other tethering component to connect a worker to an anchoring point such as a building.

These devices may have particular components described further herein which may interact to facilitate functionality across the system as a whole.

### Device: Suspension

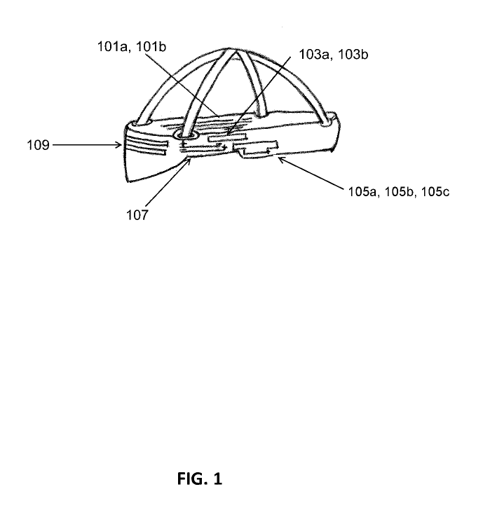

Referring to FIGS. 1-5, an interchangeable safety harness suspension (suspension 100) for use inside of a protective helmet or similar headgear is disclosed. The protective helmet or headgear may be a hardhat. While the term suspension is used, this term should be understood to encompass, but not be limited to, a safety harness/suspension for a head. A head protection system may be comprised primarily of: a shell (helmet), a headband (safety harness), and a suspension system (suspension). The safety harness and suspension may be referred to as a “suspension” herein. These components interact in order to facilitate a requisite level of protection. For example, the shell itself may cause any force of impact to be spread across the surface area of the shell. Next, the harness may separate the wearer's head from the shell such that there is an air gap between the shell and the wearer's head. This air gap may further attenuate any impact to the shell. For example, if a shell sustains an impact, the shell may flex inward and the straps of the suspension may stretch. The air gap may accommodate the shell flex and stretching of the straps. By this mechanism, the shell/suspension/air gap may prevent the wearer's head from contacting the shell, thereby reducing the risk of injury from an impact.

Referring to FIG. 1, a suspension for a helmet, the suspension having a plurality of sensors is shown, according to various embodiments. The suspension 100 may have a band and top straps. The suspension 100 may be constructed of a plastic or other suitable material(s). The suspension may have an integrated flexible or stretchable circuit board (integrated circuits 107) (though non-flexible circuit boards should likewise be understood as within the scope of this disclosure). The circuit board may further comprise one or more antennas 101. The suspension may have a micro-strip antenna 101a and GPS, SAT 101b. The suspension may include an IMU CPU 103a and battery 103b. The suspension may have a plurality of sensors (175a, 175b), one or more power sources (such as battery 103b), one or more Light-Emitting Diodes (LEDs) 105a, one or more video cameras 105b, one or more antennas 101a, one or more Global Positioning Systems (GPS) or Satellite units 101b, one or more induction charging coils 109, one or more integrated circuits 107, one or more IR sensors 105b, one or more speakers 105c (for example, temple bone conduction speakers), one or more microphones (for example, a bi-directional noise cancelling microphone), and one or more batteries. The suspension may be provided with a Universal Serial Bus (USB) port, memory card, Service Set Identifier (SSID), signal device, etc. The camera 105b may include wireless High Definition Multimedia Interface (HDMI) for recording high-definition video and high definition audio (the camera may further comprise one or more microphones). A USB port may be provided in/on the suspension and used to charge and/or transfer data between one or more devices (including a computer).

The suspension and devices may be required to withstand harsh conditions, including dust, wind, water (including complete submersion), salt spray, impact, solar radiation, and the like. In other words, the housing for the hardware/firmware may be configured to maintain, in various embodiments, active accurate measurement capabilities while surviving the possibility of complete submersion of water/mud salt spray, blowing sand or dust, and solar radiation. Therefore, elements should be understood as integrated within the band in such a way as to prevent any entry of foreign bodies into the components. The camera, microphone, and/or any other sensor or LED(s), data transmission mechanisms, etc. may be secured in a protective housing, in various examples of embodiments. A PZI nano-coating may likewise be provided. The suspension and devices may also be required to perform for long periods of time, for example, at least ten hours a day.

While a suspension for use with a construction helmet may be described, the suspension may be used with other helmets, such as those for climbing, rafting, horse jockeying, biking, playing sports, or other situation in which head protection is desirable.

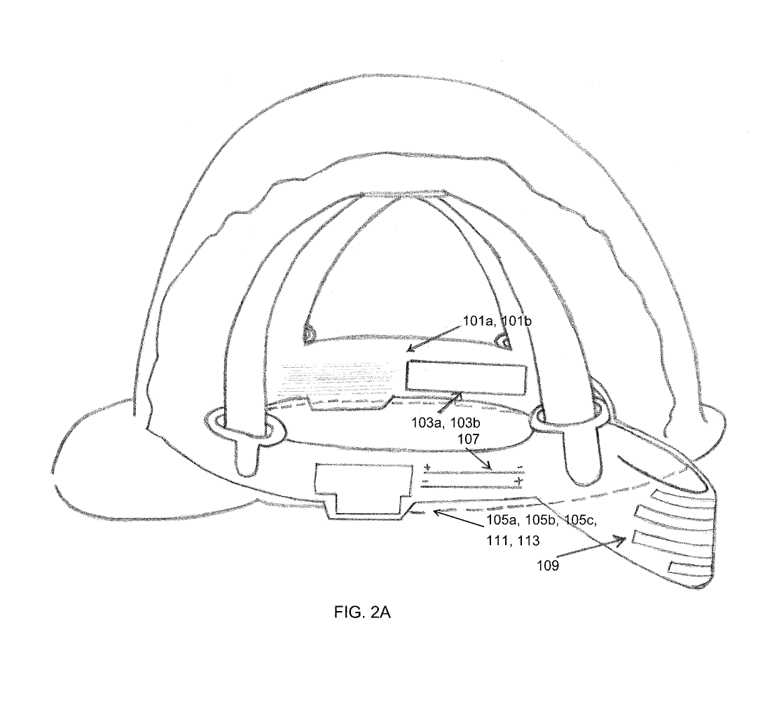

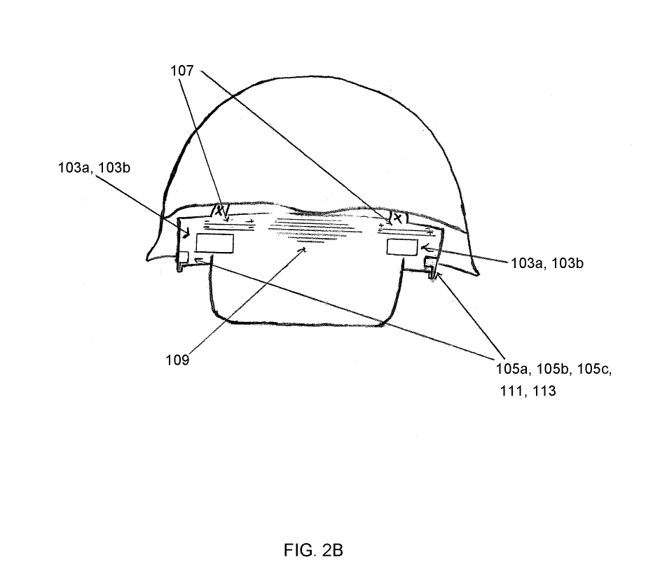

FIGS. 2a and 2b show the suspension provided within a helmet. In FIG. 2a, the suspension is shown from a side in cross-section through a helmet. In FIG. 2b, the suspension is shown from a rear view in cross-section through a helmet. The suspension and components may be provided into the helmet by way of the suspension. The suspension may allow for the helmet to protectively couple to the wearer's head. Again, the suspension components may be located throughout the suspension band. The components may include an antenna (e.g. microstrip antenna) 101a, GPS/SAT (satellite) unit 101b, an IMU (inertial measurement unit) CPU (central processing unit) 103a, a battery 103b, integrated circuits 107, LEDs 105a, camera (e.g. a video camera) and IR (infrared) sensor 105b, microphones 111, speakers 105c, and induction charging coil 109.

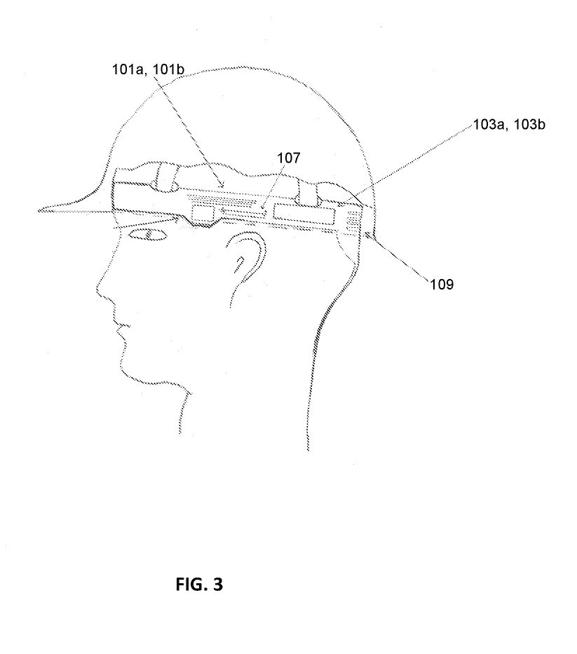

FIG. 3 shows the suspension 100 and helmet on a wearer's head. The suspension components may be provided next to the user's head, in various embodiments of the invention. The suspension components may be suitably distributed to accomplish the component's purpose. For example, the LEDs 105a, video and IR 105b, speakers 105c, and microphone 111 may be seen positioned between the wearer's eye and ear. The antenna 101a, GPS and satellite 101b may be positioned more towards an upper part of the band. A wireless charging conduction coil 109 and/or other charging apparatus such as USB may be located more toward the back of the wearer's head. The computer 103a may be seen between these components and may be connected by flexible integrated circuitry 107, according to various embodiments of the invention. A flexible circuit board (PrCBs (program requirements control board)) 101a, 103a and stretchable circuits 107 embedded into and through-out an interior of the suspension may likewise be used. The interchangeable interior suspension 100 may include metamaterials based Ku-band satellite antenna subsystem module (ASM). The suspension may further include mircostrip GPS and a number of sensors provided embedded in and/or stacked through-out the flexible circuit boards (PrCBs) in various embodiments. While a particular arrangement may be disclosed, various re-arrangements and positioning may be contemplated as within the scope of the invention.

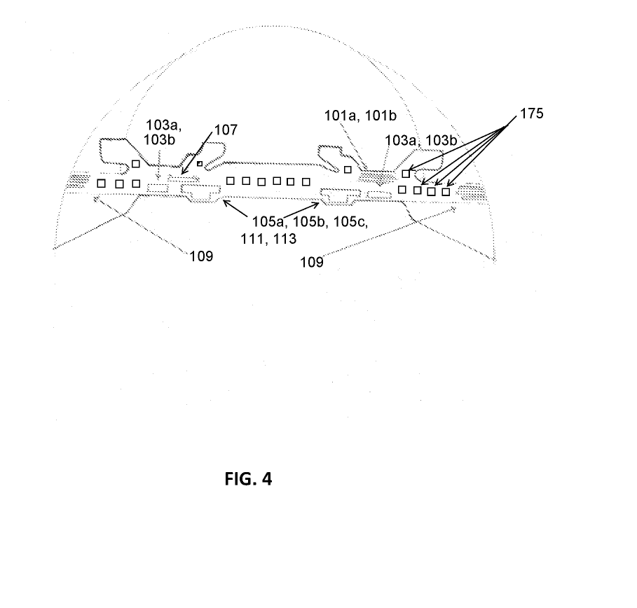

FIG. 4 shows an additional perspective of the suspension 100. This perspective can be understood as taken from a front of the suspension which wraps around a front of a wearer in various embodiments of the invention. Therefore, an interior surface of the suspension may be seen. The speakers 105c may be seen flanking the front of the suspension. In addition, the LEDs 105a and microphone 111 may be seen more towards a front of the suspension. In various embodiments, this positioning may allow for visibility using the LEDs 105a and functionality of the speakers 105c and microphone 111. In addition, copper pads 175a or other suitable EEG (electroencephalogram) monitoring feature may be provided along the interior surface of the suspension in various embodiments.

The LEDs 105a may, in an alternative embodiment, comprise a snake-like, retractable profile. This configuration may allow for LEDs to be provided in a retractable, telescoping tube affixed to a side of the suspension. The user may reach up, and pull the telescoping tube forward for provision of more direct light onto a subject. The LEDs 105a may be provided to sufficiently illuminate darkened work areas.

A number of sensors 175b may be embedded in the suspension 100. In various embodiments, the sensors 175b may be embedded in the band 102a or stacked throughout suspension 100. The suspension 100 may have embedded circuitry 107. The embedded circuitry may comprise flexible or stretchable circuits. This suspension 100 may be comprised of a plastic or other like multimaterial. Custom molding can provide an aesthetic surface. An EMI shielding material may also be provided. In addition, shielding material may serve as the core to protect the wearer of the digital safety-harness/suspension from any electrical magnetic or like interference that may or may not cause bodily harm. The digital safety-harness/suspension 100 sits upon the wearers head in a snug manner and conform to their wearer's heads in a safe secure manner. The size can be determined, in various embodiments, by the wearer head circumference divided by pi.

The suspension 100 may include one or more textile straps 102b. The textile straps may act as a shock absorber and may temporarily prevent an object from continuing or being in force or effect, which is used to regulate deceleration when the end of the maximum stretch of the textile straps is reached. Textile suspension straps 1022b may be used in conjunction with the digital safety-harness as a temporary deprivation or interruption of position, power, and/or privilege. The effect the safety harness has on the body after the retractable locks are in place may cause a yoyo-like effect that prevents the wearer from falling. The textile suspension straps may also serve as load bearing points to stabilize and balance the protective helmet or similar headgear during everyday use. Textile suspension straps 102b may be fabricated from “TNG fabric.” TNG fabric may be made from a silvery textile coated with nanorods and a silicon-based organic material. When four pieces of the textiles are together and pressure is applied down on the textile, the textile may captures the energy generated from the pressure. The textile may immediately pump out that energy, which can be used to power the suspension hardware including light-emitting diodes and/or other electronics. The textile suspension straps according to various embodiments may include the webbing unique to custom design, and lock into place on the safety-harness by a locking mechanism and or locking hardware.

The term “suspension” should be understood to encompass not only the suspension structure (band 102a, sweatband, straps 102b, etc.) but the sensing, data transmission, lighting, radio transmission, computing features, communication features, power features, radio and data transmission features, tags, and the like coupled, embedded, affixed or otherwise provided thereon/therein.

### Suspension: Sensors

The suspension 100 may include a plurality of sensors 175b. The sensors 175b may comprise an IMU (or inertial measurement unit). The sensors 175b may be part of a MEMS unit (INS/GPS, IMU). The sensors may further comprise a GPS sensor. The sensors 175b may further comprise a light sensor for triggering a self-illuminating LED (reduced light levels are measured and the LED illuminates. The sensors 175b, in various embodiments, may include a GPS unit 101b or other suitable location mechanism. The sensors 175b may include body condition sensors for measurement of a wearer's movement (e.g. measurement of kinesis and all embodiments). The sensors 175b may be coupled to a computing unit 103a for regular transmittal of sensor readings. The sensors 175b may be distributed throughout the suspension (e.g. throughout a suspension headband 102a) as shown in FIGS. 1-5. While the sensors are described herein in connection with the suspension, it should be understood that similar sensors may be provided in other devices such as the harness 200. In various embodiments, similar sensors may likewise be provided as part of gloves 300 and/or earbuds 400.

As a summary, the sensors 175b may include but are not limited to: a gyroscope, accelerometer, magnetometer, pressure-sensor, & Infrared-sensor (IR) in various embodiments. The use of a highly accurate micro-technology (micro-PNT (position, navigation, and timing)), (MRIG (Microscale Rate Integrating Gyroscope)), (PASCAL), (CSWaP), and (QuASAR) (positioning systems) program solutions for absolute positioning, navigation, and timing in the absence of GPS capabilities may in various embodiments be provided in the state of the art MEMS (micro-electrical mechanical systems) TIMU (timing inertial measurement unit) for the suspension 100. A single chip wireless-timing-inertial-measurement-unit (WTIMU) may be provided. The invention may include a digital camera 105b suitable for streaming audio/video and a wideband data digital 2-way radio communication system 113 with self-illuminating LEDs 105a, provided within the interchangeable interior suspension 100. Data may be provided by the devices including location (for example, GPS or relative location), health vitals (such as heart rate, respiration, temperature, etc.), external temperature, humidity level, etc. The device may also feature a master timing clock which may allow for simultaneously measuring tracked movement and timing such that the data may be correlated or combined.

The sensors 175b may generally detect wearer conditions (movement, biometrics), wearer location (relative or positioning technology), and environmental conditions (video, temperature, etc.).

The sensors 175b may detect wearer conditions. To this end, a MEMS unit may be integrated into the suspension. Sensors 175b may detect certain wearer conditions or measurements relating to a wearer's physical state. In addition, atmospheric conditions relating to the wearer's environment may be measured. The 9 axis measurements of a wearer's movement may be measured using suitable sensor(s) 175b (e.g. 9 axis inertial movement including 3-axis gyroscope, 3-axis accelerometer, and 3-axis magnetometer). A pressure sensor and infrared sensor may likewise be provided. A resonator may also be provided. In various embodiments, an atom-based inertial sensor may be provided for extended operation. More broadly, the full parameters of Kinesis, including Orthokinesis, Klinokinesis, (i.e. Kinesis and all embodiments) may be measured. In addition, immediate atmospheric environment condition may be measured.

Accelerometers, gyroscopes, and magnetometers may all be integrated within a sensor unit 175b for measurement of wearer movement. Head impact telemetry systems may be used to determine if an impact to a wearer's head has occurred. EEG monitoring mechanisms 175a may likewise be provided (in other words, brain-wave monitoring). This monitoring may allow for the classification of certain mental states as described more herein.

Worker environment and status may be monitored. Changes in body mass, percentage of body fat, heart rate, ECG (electrocardiogram), respiration rate, core temperature, skin temperature, accelerometer, and body position may all be data points acquired in some manner (by sensors 175a, 175b or other means) using the devices, system, and method disclosed herein.

Temperature sensors may also be provided to monitor wearer and/or environmental temperatures. Sensors for capturing other biometric data such as pulse, oxygen, etc. may be contemplated as within the scope of this disclosure. Apical pulse may be used along with breathing rate.

Sensors 175b may be provided to evaluate environmental conditions. For example, accurate measurement of the ambient temperature/humidity relevant to the wearers immediate environment may allow for the evaluation of whether a working condition is safe.

The sensors 175b or suspension components may detect wearer location. The suspension may be equipped to report positioning information. GPS systems 101b (or GPS backup/alternative systems such as STOIC) may be used to provide wearer location. Other suitable mechanisms for reporting user location may be contemplated as within the scope of this disclosure. Sensors 175b including MEMS-based inertial systems for measuring heading, orientation, and position may be used (e.g. sensors such as the Spartan NavEx). Alternatively, a NEMS (nano electrical mechanical system) may be used.

The prescribed movement objects the sensors 175b may measure may be split into three different groups of movement: (1. Translation Movement, 2. Rotation Movement, & 3. Inertial (movement) Measurements Unit (IMU). These three groups may then be broken down into nine basic ways any object can move. These nine basic movements are classified as “Degrees of Freedom” (DOF). Within the three groups of Translation, Rotation, & IMU Movements, any object is free to translate into 3 out of 9 basic movements or Degrees of Freedom (9 axis): 1. Translation Movement=3 DOF=(1, Forward & Back), (2. Up & Down), and (3. Left & Right); 2. Rotation Movement=3 DOF=(1. Yaw), (2. Pitch), & (3. Roll); 3. Inertial (movement) measurement Unit=3 DOF=(1. Velocity), (2. Orientation), & (3. Gravitational Forces/Electromagnetic).

Known indoor positioning systems may be contemplated as within the scope of this disclosure for use with the disclosed device, system, and method. While a brief synopsis of potential technologies is provided, known suitable indoor positioning systems should be contemplated as within the scope of this disclosure.

Suitable indoor positioning systems may include the use of radio waves, magnetic fields, acoustic, signals, or other sensory information collected by mobile devices. These could include distance from anchor nodes having known positions, magnetic positioning, or dead reckoning positioning (typically, trilateration may be necessary to determine position). Locator Nodes or tags may be used to provide positioning relative to tagged locations throughout a site (e.g. using a NFC (near-field communication)/RFID (radio frequency identification) reader responding to tags). Bluetooth (e.g. beacons using proximity sensing), wireless technology (e.g. wireless proximity sensing), or LiFi (Light Fidelity using lightwaves for positioning encoding) could also be used to provide wearer positioning information. Ultra Wide-Band technology could also be used. A barometer may also provide information for elevation evaluation. Optical tracking of markers such as a QR code (Quick Response Code) could provide relative positioning insight. A variety of suitable known indoor positioning systems (IPSs) may be used and should be contemplated as within the scope of this disclosure.

The suspension may also include one or more light sensors and one or more light-emitting diodes (LED(s)). The light sensors may detect when the worker is in limited light (visibility) conditions. When the condition is sensed, the light-emitting diode may automatically turn on. The LED may either increase in brightness relative to visibility (i.e. detected light levels) or fully turn on and off relative to a detected brightness threshold.

Again, while these features are described in conjunction with a suspension, the same or similar sensing, data transmission, lighting, radio transmission, and other features may be embedded, coupled to, or otherwise provided for in a harness. In addition, similar features may be, for example, sensors, lighting, and data transmission features may be provided for in devices such as one or more gloves or earbuds.

### Suspension: Computer

The suspension may include a CPU (central processing unit) or computational device (computer) (e.g. computer unit) 103a. The computer unit may request and accept sensor 175b readings, request and accept communication connections, connect to one or more wireless networks, and transmit data over a network, in various examples of embodiments. The computer may be embedded within the suspension 100 and connected to the sensor components by embedded flexible (possibly stretchable) circuitry 107.

The computer unit may include onboard memory. The onboard memory can, in various embodiments, be a flash-type (solid state) storage medium. The onboard memory may include a suitable capacity to store identification information relating to the wearer or suspension identifier. The memory may also include temporary memory such as RAM (Random Access Memory) for accomplishing the features described herein. The computer unit 103 may include data transmission mechanisms described herein.

The processor may be suitably powerful to accomplish the requirements; however, it may balance power requirements to allow for the use of the suspension during a typical workday.

The network may include a secure protective busing for wireless nano processors for the differing tasks. The network and computer may be suitably configured for streaming high-definition video into a secure cloud for multiple uses.

Again, while the computer (computing features) including memory and storage is described in conjunction with a suspension 100, the same or similar computing features may be embedded, coupled to, or otherwise provided for in a harness 200. In addition, similar computing features may be provided for in devices such as one or more gloves 300 or earbuds 400.

### Suspension: Battery and Charging

The suspension 100 may include a battery 103b for operation of the suspension components. The battery 103b may be charged by way of a suitable charging technology. The battery may be a solid state lithium ion battery.

In various embodiments, wireless charging may be used and an induction coil 109 provided on, coupled to, or embedded in the suspension 100. The suspension 100 may have a charging mechanism 109 designed to be used with the charging station disclosed herein, in various embodiments.

Inductive Power Transfer (IPT), Inductive Coupling, and/or Resonant Power Transfer may be usable with the devices disclosed herein. Each of these terms may be understood to describe a similar fundamental process—the transmission of energy from a power source to an electrical load, without connectors, across an air gap. The devices may therefore use a wireless power system having essentially two coils—a transmitter and receiver coil. The receiver coil may be provided in the device (suspension, body harness, glove, earbud, etc.), while a transmitter may be provided on a charger.

The device and system may use magnetic wireless inductive charging, in various embodiments. The device may also be provided with one or more standard USB ports for direct charging of the suspension, according to various examples of embodiments. For example, wireless charging and/or USB multifunction charging data cables may be provided.

Again, while the power features including power storage (batteries) and charging features (inductive, usb, etc.) are described in conjunction with a suspension 100, the same or similar power features may be embedded, coupled to, or otherwise provided for in a harness 200. In addition, similar power features may be provided for in devices such as one or more gloves 300 or earbuds 400.

### Suspension: Communication

The suspension 100 may be further comprised of one or more communication elements. The communication elements may comprise one or more speaker(s) 105c and microphone(s) 111. Communication via speaker(s) and microphone(s) may present special challenges given an application for head protection, especially in construction. As can be imagined, construction may involve excessive amounts of noise, potential for damage, and the like.

The speaker(s) 105c may, in various embodiments, be temple bone conduction speakers (bone conduction headphones). The speaker may be suitably positioned around the head suitably close to enable proper function on the speakers.

In various embodiments, the suspension 100 may also comprise a microphone 111. The microphone 111 may be a bi-directional, noise-canceling microphone 111. The microphone 111 may be suitably positioned in the suspension to enable easy communication. Audio transmission may be “hands free” allowing the worker to continue to work even while communicating. In various embodiments, the “hands free” functionality may be facilitated by use of a keyword. The keyword may cause the system to wake up and begin streaming the audio.

A digital camera 105b may also be provided in various embodiments. The digital camera 105b may have a form factor similar to those provided in a standard smart phone. The digital camera 105b may be used to capture images or video. The digital camera 105b may begin streaming video upon an audio command, for example, by the wearer. Alternatively, the camera provided on the suspension 100 may be accessed remotely by a supervising individual for live access to worker progress. The video camera 105b may also be used to capture and identify QR codes or other suitable tags provided throughout the project site. In this embodiment, known QR code interpreting software may be used in the system.

A 3D/4D IR (infrared) scanner (e.g. in connection with the camera) 105b may be also provided in the suspension 100 and considered within the scope of this disclosure. The scanner may be used to capture 3-D/4-D IR scans for ERP models. The use of 4-D scanning in 4-D interactive GPU (graphics processing unit) models may enable the architects, designers, contractors to clients of heavy industry projects, to visualize the entire duration of a series of events and also display the progress of construction/heavy manufacturing activities through the lifetime of the project, in various embodiments. The system may allow for three-dimensional graphics of project production.

Suitable sensors/readers such as an RF (radio frequency) reader may be provided (e.g. in connection with the camera) 105b to identify tags provided on equipment or locations throughout the jobsite.

Again, while the communication features including speakers, microphones, cameras, IR scanners, RF readers, and the like are described in conjunction with a suspension, the same or similar communication features may be embedded, coupled to, or otherwise provided for in a harness 200. In addition, similar communication features may be provided for in devices such as one or more gloves 300 or earbuds 400.

### Suspension: Radio and Data Transmission

The suspension 100 may also include wireless communication functionality. The wireless communication functionality may be enabled by a two-way radio 113, data transmission mechanism provided in the computer unit 103a, or separately provided on the suspension 100. The suspension 100 may be programmed on deployment (for example, at the beginning of a project or workday) to communicate with other devices being worn by other members of a communication group (e.g. other workers in a particular area of a jobsite). The programming may allow for connection to a particular channel. The wearer may be able to connect to communicate with other workers/wearers on a particular channel by way of a keyword which is detected by a microphone 111 and interpreted by the data system (i.e. processor, server, etc.) 103a (locally), 501 (remotely), according to various embodiments.

The wireless communication functionality may be equipped to stream sensor data, live video, and audio. The wireless communication functionality may be enabled by one or more of the following technologies: advanced wireless ad hoc-networking, WiFi, Bluetooth (Bluetooth LE), mobile (e.g. LTE), Zigbee or other mesh-radio networks, SRF and point-to-point radio links, UART or serial lines, SPI or I2c wired buses.

In an alternative embodiment, EEG may be used to communicate between crew members or across the system disclosed herein. The EEG apparatus may be integrated within the suspension. The EEG apparatus may facilitate measuring levels of brain activity while performing various tasks. The EEG apparatus may also allow for stimulate regions in the brain to gain a more productive worker or wearer of the device. Data regarding worker brain activity may be transmitted using the methods disclosed herein. Stimulation of brain regions may be activated using the dashboard or other suitable methods disclosed herein. EEG may allow for monitoring of high and low brain activity and monitoring of worker productivity. Monitoring EEG activity may allow for the development of worker baseline EEG productivity. Provision of a baseline EEG level may allow for customization of a worksite crew relative to EEG activity levels. EEG may correlate with job proficiency and allow for insight into worker skill proficiency.

The speakers disclosed herein, for example, one or more temple bone conduction speakers 105c may be used to stimulate EEG activity. Copper pads 175a may likewise be provided in the suspension.

Again, while the radio and data transmission features are described in conjunction with a suspension, the same or similar radio and data transmission features may be embedded, coupled to, or otherwise provided for in a harness. In addition, similar radio and data transmission features may be provided for in devices such as one or more gloves or earbuds.

### Suspension: Tag

The suspension may have one or more QR codes, RFID tags, or the like (“tag”) for ease of tracking of the device(s). In various embodiments, an identifier such as a tag (e.g. QR code or RFID tag, etc.) may be provided on the suspension 100 for ease of identification.

Once the suspension is identified, various information may be correlated with the suspension such as worker identification data. This worker identification data may include various information provided into a human resources portal.

Again, while the tag is described in conjunction with a suspension, the same or similar tag may be embedded, coupled to, or otherwise provided for in a harness. In addition, tags may be provided for in devices such as one or more gloves or earbuds. The tag may also be used as described further herein in connection with resources and equipment.

### Suspension: Use

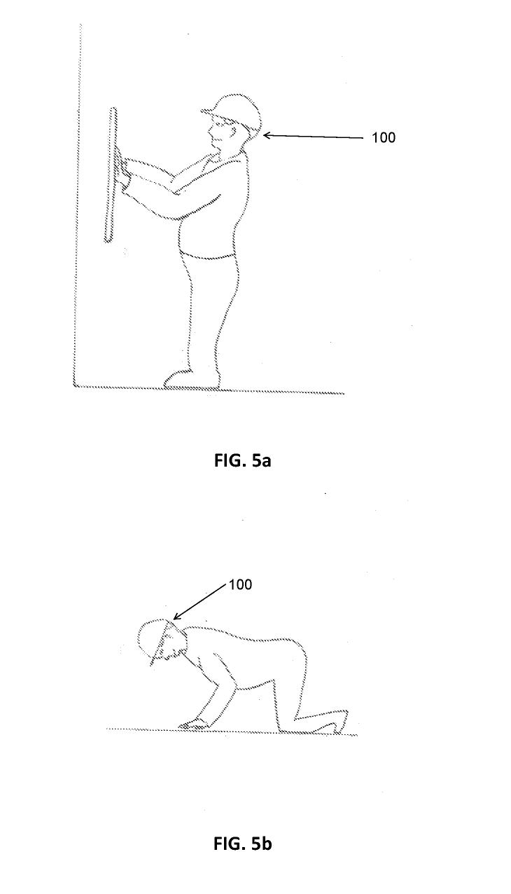

FIGS. 5a and 5b show a worker having a protective helmet on containing the suspension 100 according to various embodiments.

FIG. 5a shows a worker in an upright position wearing a protective helmet having the disclosed suspension 100. In this position, the suspension 100 (including the suspension computer and sensors) may continuously register sensed user conditions (upright, arms extended, biometrics), location, and other metrics related to the worker's behavior. For example, if the worker is detecting something wrong with the surface in front of him, and wishes to notify other team members, the worker may speak a keyword to connect via two-way radio 113 to other workers. In another example, a supervisor may remotely access the worker's sensors 175b or camera 405b to survey the situation.

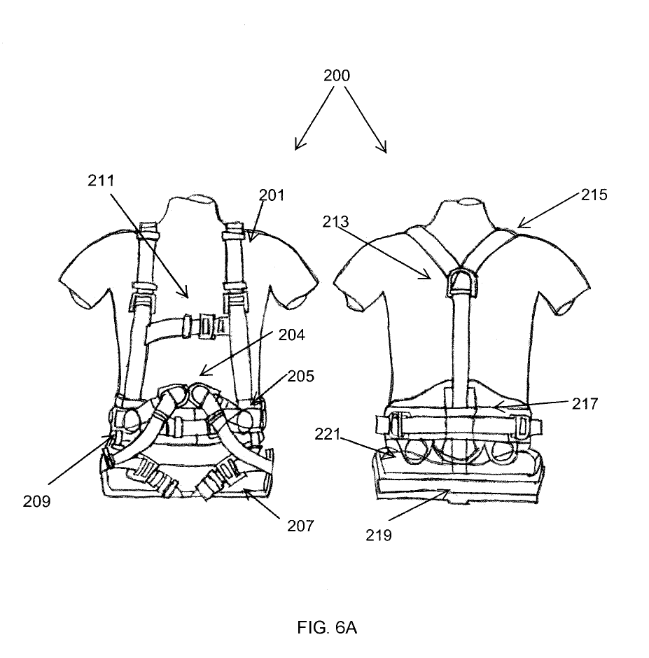

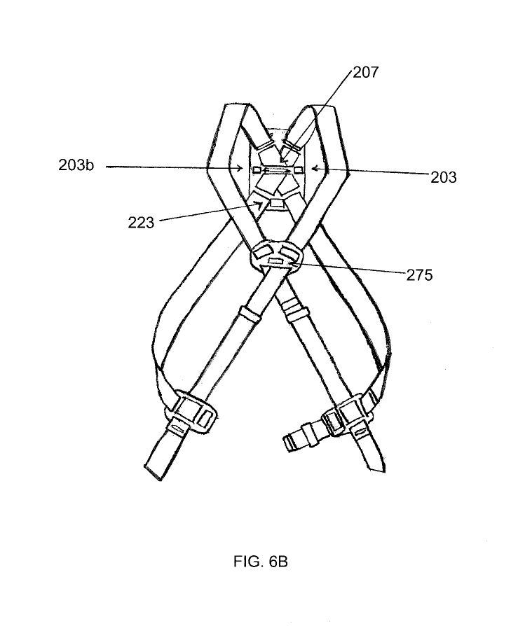

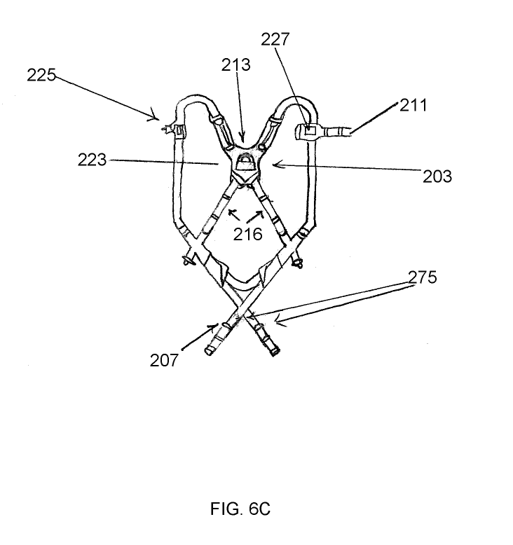

FIG. 5b shows a worker on hands and knees wearing a protective helmet covering the disclosed suspension 100. Again, in this position, the suspension 100 may continuously register sensed user conditions (hands and knees, other biometrics), location, and other metrics related to the worker's behavior. As another example, in looking down, the worker may be experiencing reduced visibility conditions, for example, reduced lighting. Therefore, an LED 105a provided on the suspension may automatically detect these conditions (using sensors 175b) and turn on the LED 105a to remedy the reduced visibility conditions. As another non-limiting example, the worker shown in FIG. 6 may be experiencing a health or emergency condition—for example, a fall or impact. The sensors 175b provided on the suspension 100 (or other device such as the body harness 200, earbuds (ear plugs) 400, or glove 300 further described herein), may sense an elevated biometric condition (for example, by comparing the detected conditions to baseline data), producing an alert. This alert may be provided to a relevant recipient, allowing for communication or attention with the troubled worker.

In various embodiments, the device(s) may advantageously allow for an accurate measuring tool for gauging strikes to the head or other abrupt movement by the wearer while performing a task. The device may also advantageously allow for an accurate measurement of vibrational strains on the wearer.

### Device: Harness

FIG. 6A shows a front and back view of a body harness 200, according to various embodiments of the invention. While the term harness is used, the term should be understood to encompass but not be limited to a safety harness/suspension for a body (as distinguished from a head). FIG. 6A shows an example harness having a number of harness components. The components may include an adjustable shoulder strap 201, positioning belt 203, abdominal cushioning pad 205, adjustable thigh strap 206, D-ring positioning 209, adjustable chest strap 211, attachment D-ring 213, elastic shoulder strap 215, cushioned back pad 217, and tool holders, 221. The harness may be provided on the body of a worker and may be designed to tether a worker to a stable surface for protection in the event of a fall, for example.

As previously disclosed, the sensing, data transmission, lighting, radio transmission, computing features, power features, communication features, radio and data transmission, tags, and other components described in connection with the suspension 100 may be included embedded, coupled to, or otherwise provided for in a harness 200. Therefore, FIGS. 6B and 6C show various embodiments of a harness having a number of features.

The term “harness” should be understood to encompass not only the harness structure (chest straps, leg straps, belt, etc.) but the sensing, data transmission, lighting, radio transmission, computing features, communication features, power features, radio and data transmission features, tags, and the like coupled, embedded, affixed or otherwise provided thereon/therein.

FIG. 6B shows a first (front) perspective of a harness 200 according to various examples of embodiments. The harness may include LEDs 225 and sensors including an LED sensor 227, a two-way radio 223, an IMU CPU 203, integrated circuit 207, and battery 203b, according to various examples of embodiments. FIG. 6C shows a second (back) perspective of a harness according to various examples of embodiments. The harness may be shown having a D-ring for attachment 213, one or more LEDs (including light sensors 227) 225, chest straps 211, two-way radio 223, IMU CPU 203, sensors 275, and leg straps 206. The LEDs 225 may be similarly provided in the harness for enhanced visibility. The LEDs may be self-illuminating LEDs.

The body harness 200 may, in various embodiments, have similar sets of sensors embedded throughout as the suspension and provide similar safety and other data regarding the wearer. In addition, the body harness may have similar sets of wireless data transmission communication, power and charging features 203b which may include an inductive charging coil, communication components, computing components, sensing devices, and similar components. In addition, flexible circuits and materials may likewise be used. In other words, the body harness 200 may have the same components disclosed previously regarding the suspension 100 in a same or differing form factor.

Similar functionality as the suspension 100 may be contemplated as within the scope of the disclosure relative to the body harness 200. The number of components may be increased or decreased. Additional points of reference for the sensors 275 relating to movement (a number of MEMS units, for example), may allow for further details regarding worker movements and environment.

The body harness 200 or other device disclosed herein may be provided with sensors 275 for detection of temperature and humidity. One or more temperature sensors may measure ambient temperature. One or more temperature sensors may measure the wearer's temperature. Humidity and temperature sensors may be provided, for example, on a D-ring of a body harness.

The body harness may provide for two-way radio functionality. The digital two-way communication feature may work in conjunction with a two-way radio feature provided in the suspension. For example, a main box of a digital two-way radio may be disposed of in the suspension, while mechanisms for hearing (e.g. temple bone conduction speakers) may be provided in the suspension. Microphones may be provided in a suitable location on the harness, for example, near a shoulder of a wearer. The microphones may be in communication with two-way radio functionality or other suitable transmission (e.g. radio) device as described further herein.

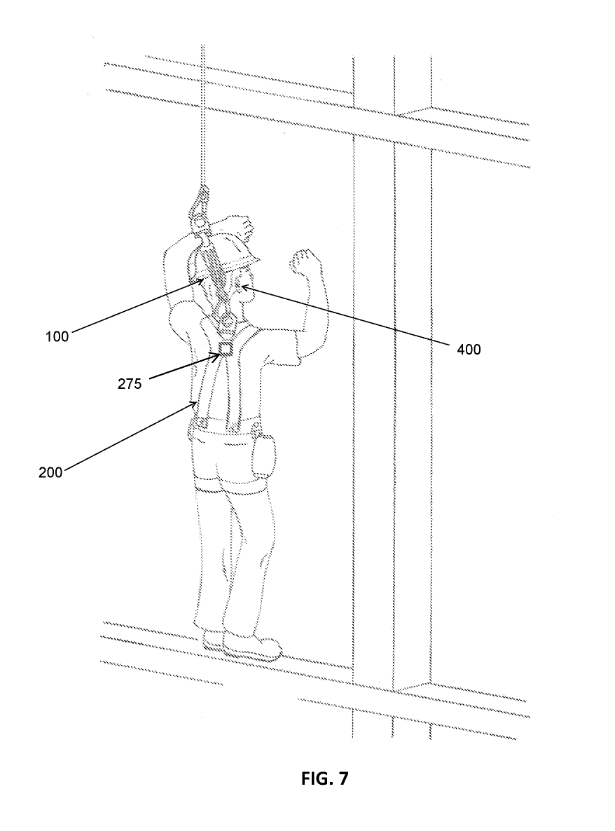

FIG. 7 shows an individual having a body harness 200 while working. In various embodiments, the harness sensors 275 may report certain condition data to the network 500 or the suspension 100 during use for transmission to the server using the system disclosed herein. In addition, an earbud (earplug) 400 for use with the system disclosed herein may be seen. The earbud may likewise transmit data to the suspension 100 or harness 200 during use for reporting of worker and environmental conditions.

### Device: Gloves

As previously disclosed, the sensing, data transmission, lighting, radio transmission, computing features, power features, communication features, radio and data transmission, tags, and other components described in connection with the suspension 100 may be included embedded, coupled to, or otherwise provided for in one or more gloves 400.

The term glove or gloves should be understood to encompass not only the glove but the sensing, data transmission, lighting, radio transmission, computing features, communication features, power features, radio and data transmission features, tags, and the like coupled, embedded, affixed or otherwise provided thereon/therein.

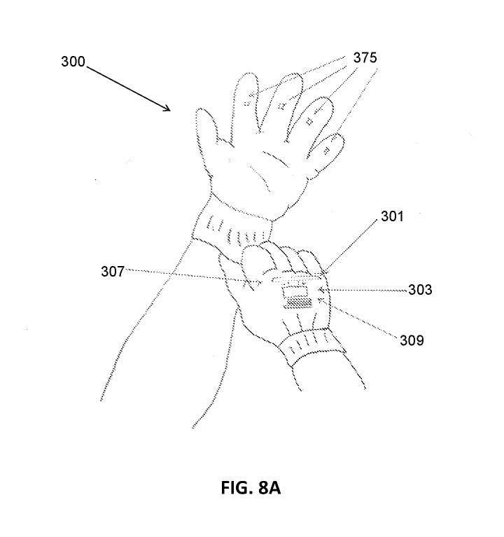

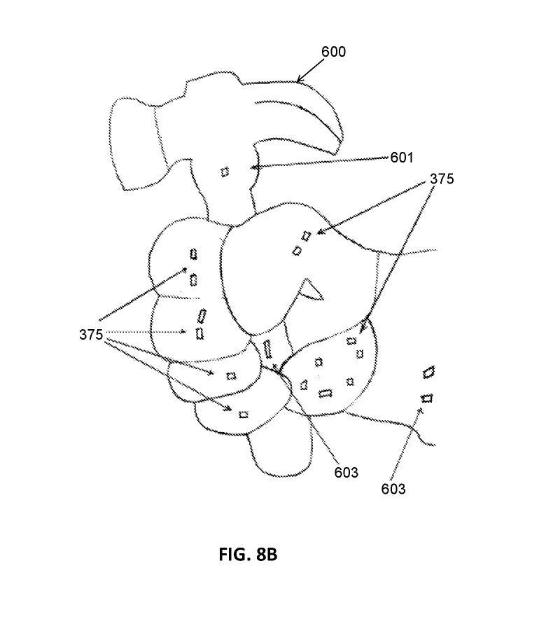

FIGS. 8a-8b show gloves 300 according to various embodiments of the invention having a number of sensors 375 for use with the system and method herein. The sensors 375 provided on the gloves 300 may be substantially passive—i.e. collecting data and transmitting to another component such as the suspension or body harness. Sensors 375 provided in the gloves 300 may be substantially similar to those provided in the suspension 100 or body harness 200. For example, MEMS devices may be deployed in the gloves. One or more data transmission mechanisms may likewise be provided, for example, in the computer unit 303. The data transmission mechanism(s) may include those provided in the description of other devices disclosed herein (for example but not limited to, Bluetooth, wifi, Zigbee, radio, etc.).

The gloves 300 according to various examples of embodiments may include sensors 375 on the front and/or back of the fingers. The gloves 300 may also include one or more LED lights 301, for example, for illuminating darkened conditions and/or indicating device status. The gloves 300 may likewise include one or more of the following: integrated circuits 307, an IMU/CPU 303a, a battery 303b, an inductive charging coil 309, and other elements as described herein regarding the other devices in the system. The gloves may also contain a “tag” 311 such as a RFID sensor, QR code, or the like. The tag may be provided for identification purposes.

FIG. 8b also shows one or more sensors or tags 601 provided on a tool, for example, a RFID sensor (other sensors or tags may likewise or alternatively be provided). This sensor and alternatives may help to identify and track tools as disclosed herein.

### Device: Earbuds/Earplugs

As previously disclosed, the sensing, data transmission, lighting, radio transmission, computing features, power features, communication features, radio and data transmission, tags, and other components described in connection with the suspension may be included embedded, coupled to, or otherwise provided for in an earbud or earplug (the term earbud may be interchangeable with the term earplug for purposes of this description).

The term “earbud” and/or “earplug” should be understood to encompass not only the earbud or earplug structure but the sensing, data transmission, lighting, radio transmission, computing features, communication features, power features, radio and data transmission features, tags, and the like coupled, embedded, affixed or otherwise provided thereon/therein.

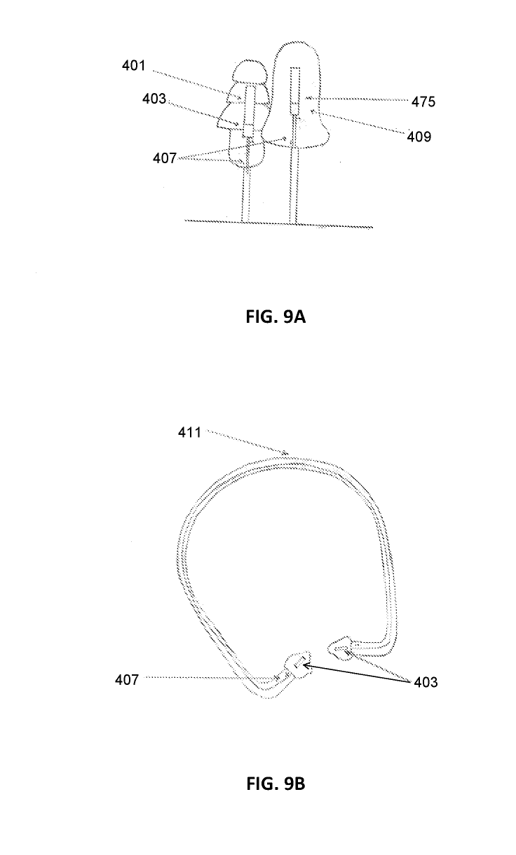

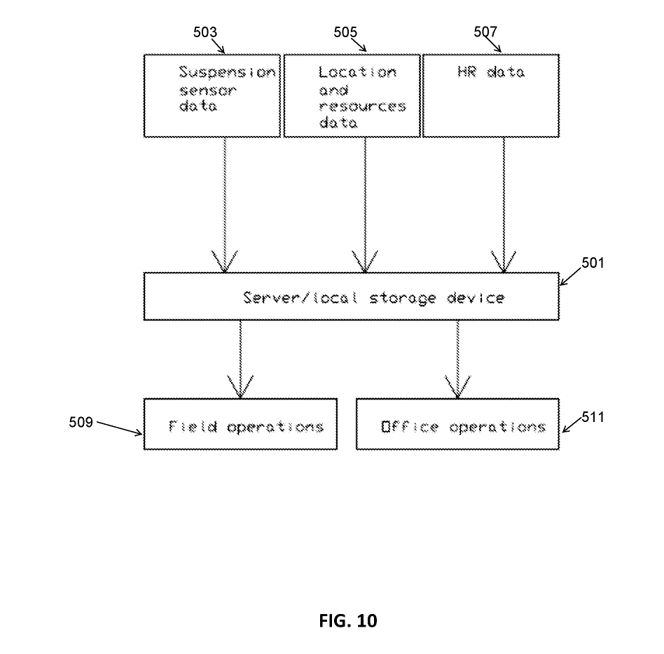

FIGS. 9a-9b show various embodiments of earbuds or earplugs 400. In various embodiments, headphones, earbuds, or earplugs (“earbuds”) may be provided having sensors 475 and data transmission mechanisms 403. This may advantageously allow for ear protection and additional data gathering. The sensors 475 may include features disclosed regarding the devices herein. The sensors 475 may include a temperature sensor. The temperature sensor may measure and report the wearer's internal temperature. The data transmission mechanism(s) may include those provided in the description of other devices disclosed herein (for example but not limited to, Bluetooth, wifi, Zigbee, radio, etc.) 403. The earbuds 400 may alternatively use a passive relay for transmission of sensor data. The earbuds 400 may transmit data to a larger device such as a suspension 100 for a helmet disclosed herein or a body harness 200. The earbuds 400 may be used for safety purposes and may include noise-reducing features. The earbuds 400 may also detect breath and heart rate. Alternatively, headphones, independent earbuds or connected earbuds, a headband, for example, a sweatband may be used. While the term “earbud” is used, any suitable device for protecting the ear and/or providing sound into the ear should be contemplated as within the scope of this disclosure.

### Device: QR Codes/Tags

The system and method herein may include using tags such as QR codes or RF tags in connection with various supplies or locations in the job site. The QR codes or RF tags may be deployed on supplies as they are brought into the jobsite. The QR codes or RF tags may be provided on walls or other locations as construction of a building progresses. In this way, the tags may provide one or more mechanisms for relative positioning. The QR codes or RF tags may be correlated with the supplies, items, or locations in a database or other suitable storage mechanism. This information may be stored on a server for active correlation with their respective items during regular operation of the system

### System: Summary

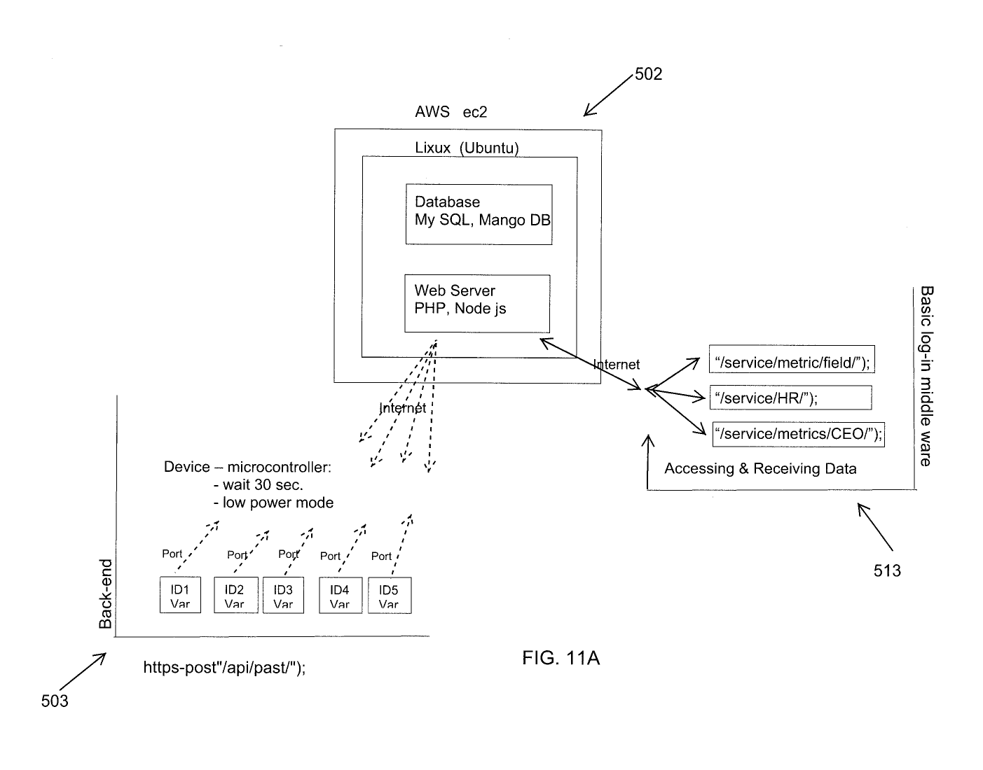

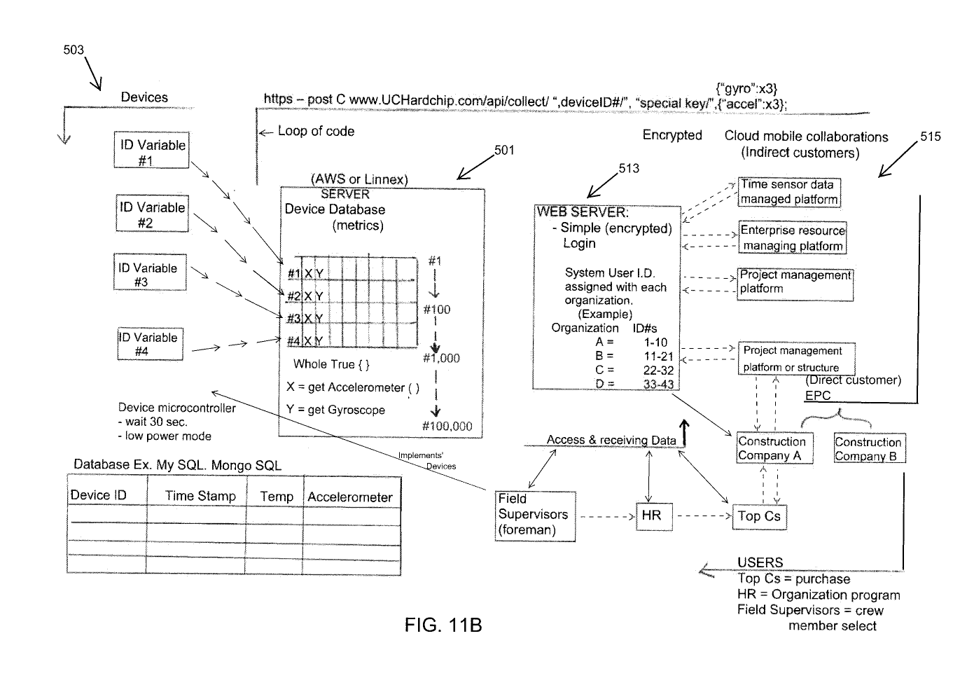

The devices, including the suspension 100, harness 200, gloves 300, and earbuds 400 may be part of a monitoring system. The system may further comprise a network (e.g. a network gateway) 500, a server 501, one or more application portals 513, and identification tags (such as passive or active RF and/or QR codes) 601. FIG. 11 illustrates a dataflow workflow, according to various embodiments. Three sources of data are shown at the top of the figure. Sensor data 503 (which may include any data from the devices disclosed herein including suspension or harness), may be provided to a server or local storage device 501 over a network 500 using the hardware and software disclosed herein.

In addition, location and resources data 507 may be likewise provided to a server or local storage device 501. The location and resources data 507 may be provided by logging the resources brought onto the jobsite. In this process, the resources may be tagged using a known tagging technology such as passive or active RF tags or QR codes 601. The correlation of the tag 601 and resource may be stored in the server or local storage device. In addition, as locations are developed across the jobsite, the location may be tagged using a similar tagging and identification means. Resources or equipment may also include the suspension 100, body harness 200, earbuds 300, or gloves 400 themselves. In other words, the system may correlate user identification with the devices they wear. This may be achieved through the tagging system or by pushing data directly onto the device.

The correlation of the tag 601 with the location may be stored in the server or local storage device 501. As the devices (such as the suspension 100) encounter the tags 601 in the jobsite, the interaction between the tag and the device may be logged. For example, as a user wearing a suspension enters a particular location and interacts (“looks” at a QR code, registers an RF tag, etc.) with a tag, the location of the suspension (worker) may be correlated. Therefore, a worker may be pinpointed at a particular location. Similarly, “tagged” resources may be interacted with by a worker. Use of the resource or equipment by a worker may therefore be logged. In a non-limiting example, a worker may use a piece of equipment in a particular location. The suspension may log the location by way of a location tag 601. Next, the suspension 100 may log the identification of the equipment by similarly interacting with a tag 601 provided on the equipment. The equipment location and use may thereby be correlated in a particular location.

FIG. 11 also shows Human Resources data 507 being provided into a server or local storage device 501. In continuing the example above, a worker's credentials may be correlated with the use of the equipment—for example, the system may be able to evaluate whether the worker is qualified to use such equipment. The Human Resources data 507 may be manually provided into the system.

The system may allow for use of the data contained in the server or local storage device 501 for various purposes. In general, these purposes may be divided into field operations 509 and office operations 511. Field operations 509 may be understood as the on the ground conditions of construction as the project progresses. Office operations 511 may be understood as the backend, including tracking time, payment, schedules, and the like.

FIGS. 11A-11B show systems for use with the system and method herein in order to accomplish the disclosed functionality. As can be seen in FIG. 11A, the acquired data 503 may be passed to a suitable storage mechanism such as a database on a server 501 for access and processing. The sensor data 503 may include one or more identification values, which may be transmitted to a web server 501 and stored in a suitable database. This data may then be optionally accessible for applications using an API (Application Program Interface) 513. FIG. 11B shows further detail of the system of FIG. 11A. Again, the devices and system provide data 503 as a number of ID variables. These may then be stored on a server 501 in a database (while relational is depicted, non-relational options should also be contemplated as within the scope of this disclosure). The web server 501 may service a number of applications 513 including project management systems. These applications may service particular organizations or third-party consumers 515 such as, but not limited to, construction companies.

### Human Resources Portal

The system in various embodiments may include a human resources portal 513 (the portal may be an application 513 provided by the server 501). The human resources portal may include various data (e.g. HR data 507) about workers. The portal 513 may likewise be updated by actual monitored production values (e.g. sensor data 503 or location and resource data 505).

The combination of entered data and live data may provide for a non-invasive accurate measuring tool for describing in quantifiable values if the wearer is performing at a high rate or low rate in relation to the cognitive and physical demands of the skilled task at hand. This information may be provided in relation to the wearer's skill-craft areas of expertise in which they specialized in performing. Further information provided regarding a worker may include average performance statistics across skilled-craft areas of expertise, training certifications (current), safety certifications (current), accidents reports (any & all), bio-metrics health-vitals that effect heart-rate and breathing-rate (age, body size, fitness level, average breathing and respiration rate, average heart rate, any heart conditions, whether the prescribed task is performed standing or sitting, medications, emotional health, average body temperature), information on all projects worked to date, and attendance score based on timely attendance for those projects and current project. Whether the worker smokes and how many years of proficiency the worker has may also be recorded. While a number of metrics are given for example, more or less may be contemplated for use with the system and method disclosed herein. Averages may be based at new hire physical aptitude examination and may be continuously updated while the worker performs various skilled tasks e.g. concrete, drywall, or wood framing. Access to the worker's health information may be limited to those with the right security clearance (e.g. those with a particular password/access level/database key).

The system herein may allow for an individual worker rating system which may be updated using the device, system, and method herein. The rating system may include an inventory of physical attributes and aptitude test results.

In various embodiments, the system may allow for smart work identification using microchip banking technology. The devices and programs disclosed herein may allow for gathering data generated through multiple nodes (or devices) within the system. Data may be transferred through a wireless connection in the nano-device (using mechanisms described herein including li-fi technology). The data may be sent through a secure data connection in the cloud. The data may also be stored. The data may include employee performance statistics, health vitals, exterior temperatures and humidity values. A UPC, tag, or other code 601 may be located on each device.

### Identification Card

The system may also comprise an identification card for workers, according to various examples of embodiments. For example, an employee (worker) may have a smart work identification card having a UPC, tag, or other code. The code may be provided on a card, in various embodiments of the invention. The card may be an identification card. By swiping or reading the card, any boss, superintendent, foreman, crew leader, partner, friend or anyone with access to the item through the web-based application may have access to certain data based on clearance. By scanning or swiping, the employee data may be seen and disclosed. This data may include location of the employee, personal health data, and/or banking data. The data may be accessible via a web-based application, thereby allowing access from any device having an internet connection.

The identification card may also contain an employee photograph and a smart code. The employee identifier may allow for easy access to employee history regarding training courses, safety training, all which may be updated with each course listed. This may all be accessible by a barcode or smart identifier. The identification card may be understood to interact with the human resources portal described herein. The identification card may be provided with mechanisms to allow for direct payment of the worker or employee. Clock in/clock out features may also be part of the card.

The card may likewise be used facilitate scheduling functionality and to correlate equipment such as the devices disclosed herein with an employee. The card may also facilitate payment of the employee/worker.

### Location System

The device, system, and method herein may allow for determination of wearer, resource, and other locations throughout the site. The sensors tagging systems 601, and internal locator mechanisms described herein may be used in various embodiments. The location system may allow for an accurate measurement tool for gauging where the wearer of a device has been. The location system and device may also provide how fast the wearer got to the location, what route they took, and determine whether other devices/workers are in the immediate vicinity.

The location system may likewise include the tracking and tagging of tools. The tools may, in various embodiments, be located using tags 601 provided thereon according to various embodiments, such as RFID, QR, IR, or other such suitable technologies. The tags may then be located by being “seen” by the suspension 100 or other suitable device. For example, the tools 600 may be “scanned,” “seen,” or “located” when the tool is provided on the jobsite (for example, using the database described herein) and subsequently “scanned,” “seen,” or “located” when encountered by a worker or other individual having a suitable device (for example, the suspension disclosed herein).

### System: Jobsite Workflow

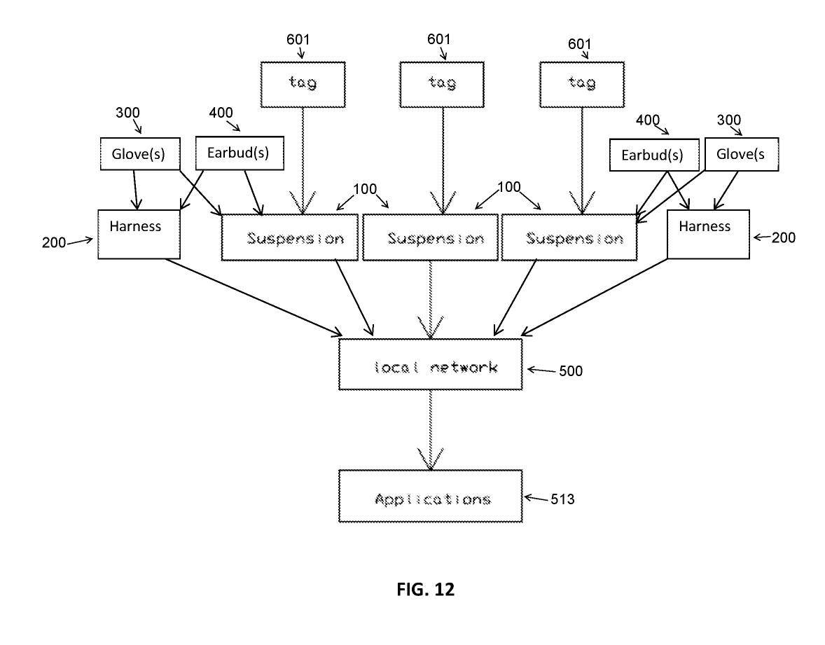

FIG. 12 shows a jobsite workflow according to various embodiments. A number of tags 601 are shown interacting with a number of devices including suspensions 100, earbuds 400, gloves 300, and harnesses 200. The tags 601 may be encountered by the worker during normal operation. For example, a worker may enter into a particular area which is identified by a tag 601. The tag 601 may be registered by the suspension 100. The presence of that tag 601 and that suspension 100 may be transmitted over a local network 500. The local network 500 may then transmit the correlation to one or more applications 513. As a non-limiting example, a jobsite supervisor may access the application 513 and be able to see where each of the suspensions 100 of FIG. 12 are located relative to the interpreted tags 601. In addition, a number of earbuds 400 or gloves 300 may sense data and transmit the data to the suspension 100, harness 200, or both, for transmission of the data in the network 500.

### System: Data Workflow

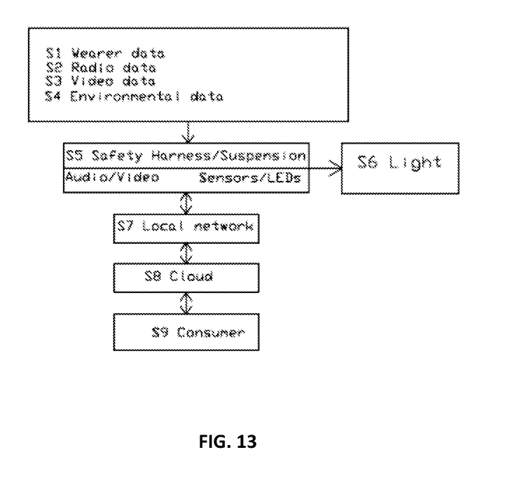

FIG. 13 shows a more detailed workflow of data obtained and transmitted according to the device, system, and method herein. Four types of data are provided into the suspension or device, in various embodiments. S1 shows wearer data—or data that is correlated between the worker and suspension. This may comprise some human resources data. The data may allow for the connection by the system of a worker to a device. In addition, wearer data may include information obtained by sensors regarding the worker's positioning, vitals, and the like. In various embodiments, this information may be obtained by the MEMS or other sensors. S2 shows radio data—or the correlation of what radio frequency the device should connect. In various embodiments, the radio frequency will be set to connect with other devices worn by workers as part of a particular team. Video or image data may likewise be obtained and provided to the device. This may include an array of video or image types, including the detection of QR or other tags. S4 shows environmental data. This data may include information regarding the location or other worker conditions. Ambient temperature, light, location, humidity, etc. may all be provided in various embodiments. This data may be obtained by the device in step S5 (suspension 100, body harness 200, glove 300, earbud 400 etc.) for local processing or interpretation, or sent to a local server for processing and interpretation. In an example embodiment, the data may cause the device to behave in a particular way, for example, by turning on the light (self-illuminating LED) in S6 or connecting to a network in S7. The local network may transmit the data to a suitable mechanism such as a cloud storage device S8. The data may be processed at that point by way of a processor provided in the cloud storage mechanism. The processed data may be transmitted back to the device over the local network for appropriate response by the device (for example, if the cloud processes “keyword” functionality, the cloud may evaluate whether the keyword was said and instruct the device accordingly; the device then may perform the keyword-enabled function). The processed or unprocessed data may be interpreted by one or more consumers, applications, etc. in S9.

### System: Device to Device Workflow

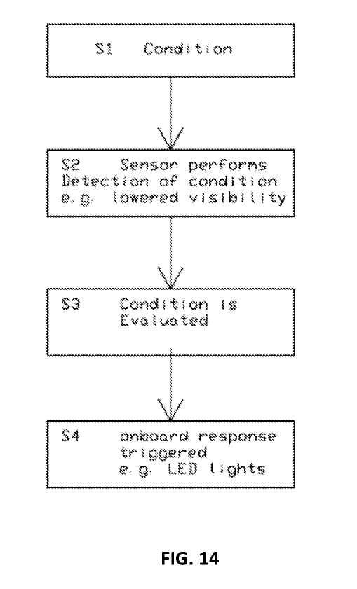

FIG. 14 shows a workflow for sensing and triggering a response within a device, according to various examples of embodiments. In S1 a condition is sensed. This may include an environmental condition, such as light levels, temperature, etc. or a noise, or movement from the wearer. In S2 a sensor performs detection of a condition. In a non-limiting example, a condition may be a lowered visibility such as lower light. In S3 the condition may be evaluated. The condition may be evaluated by an onboard process or by a remote server. In various embodiments, the condition may be compared to a known baseline data. For example, if typical light levels are a certain level, the system may evaluate whether the light level is below or above that threshold. In S4, the recognition of the condition may be translated into an onboard response. For example, in the condition of light levels below a threshold, LED lights may be illuminated. While one specific example may be provided for purposes of illustration, conditions and reactions should be understood across the varying functionality of the device, system, and method herein.

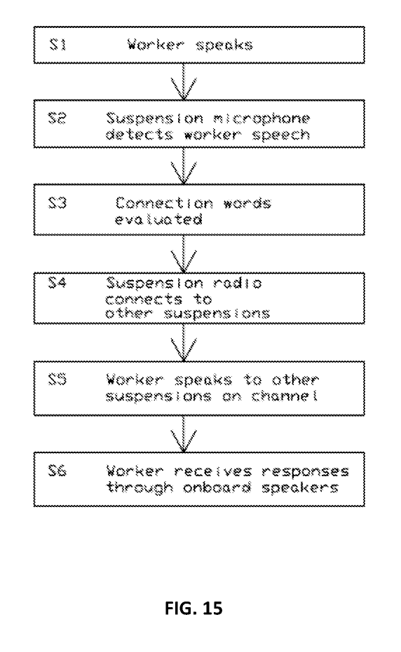

FIG. 15 shows a workflow for connecting two devices, for example, two suspensions 100, according to various examples of embodiments. S1 shows a condition where a worker speaks, creating a condition for response. In S2, the suspension microphone detects the worker speech, for example, by way of the microphone systems disclosed herein. In S3 the worker speech may be evaluated for a connection word. Such an evaluation may take place locally on the device or remotely in a cloud analysis platform. The evaluation may compare the worker's speech to a trained keyword (for example, using audio and keyword analysis mechanisms). In various embodiments, the keyword may need to be trained into the system using known training mechanisms. Evaluation of a keyword may include a suspension identifier for correlating the keyword with a particular user case. If the keyword is identified, if evaluated remotely to the device, the server may provide a signal back to the device. In S4, the suspension radio may then enable connection to the communication mechanism (for example, a two-way radio) for connection to other devices or suspensions. In S5, the worker may then be able to speak to other suspensions/devices on the radio channel, in various embodiments. The other suspensions or devices may also have their radios communicate back, where the worker receives responses through onboard speakers (for example, bone-conductive speakers as described herein) in S6.

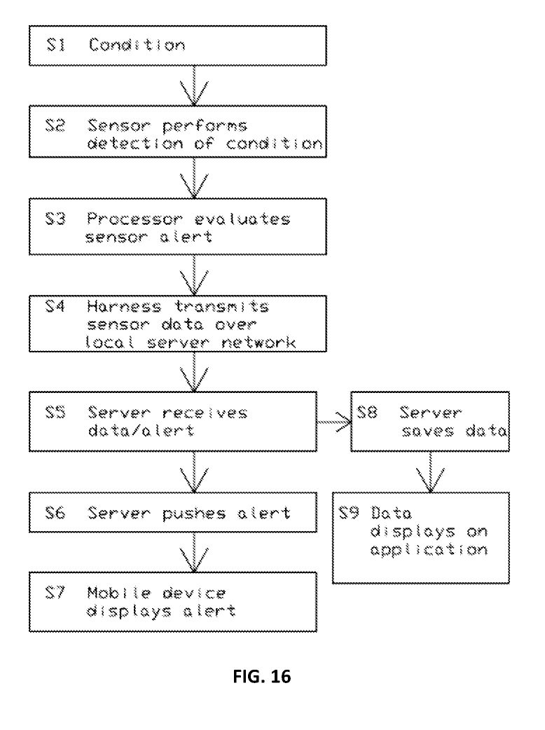

FIG. 16 shows a workflow where the device or suspension 100 communicates with a server 501. In S1 a condition occurs. This may again be an environmental or worker condition. The sensors provided within the device may detect the condition in S2. Such a condition may be, as a few non-limiting examples, a movement typical of an accident, an environmental temperature, or an elevated pulse. In S3 the processor may evaluate the sensor data. In various embodiments, the sensor data may be processed in terms of a possible alert. For example, the device may compare the sensor data to a certain threshold, such as an alert threshold of 100 degrees Fahrenheit for environmental temperatures. This comparison may be performed locally on the device or remotely (i.e. S3 and S4 may be flipped). In S4 the suspension or device may transmit either the alert or the sensor data over a local network. The data or alert may be transmitted to a server, in various embodiments of the invention. The server may receive the data or alert in S5. From here, the server may include a processor having an application programmed to perform in one or more ways relative to a data condition. In S6, the server may “push” an alert—for example, an exceeding temperature threshold—to a suitable recipient for prompt attention. In S7 the receiving device may be a mobile device having a program (for example, an SMS-enabled mobile device). The recipient would thereby be notified of the alert condition. In various embodiments, in S8, the server may instead and/or also save the sensor data (which may comprise alert data) for later review and/or analytics. The data may then be displayed on an application in S9. For example, the elevated temperature may be shown on an environmental conditions report accessible through a portal.

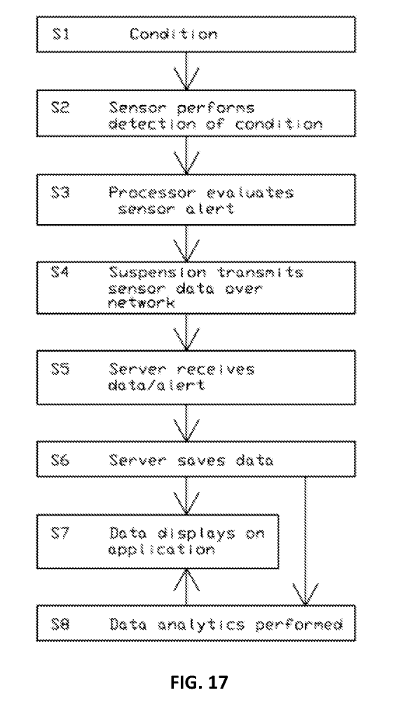

FIG. 17 shows a workflow tracking the information transmission from a device to a database to a consumer or consuming application. First, in S1, a condition may occur. In various embodiments, the condition may be an item in a particular location. The sensor may perform a detection of the condition in S2. In various embodiments, the detection may be the evaluation of a QR code, RF tag, etc. The processor may evaluate the condition or whether an alert should occur in S3. This evaluation may occur locally on the device or remotely (i.e. S3 and S4 may be flipped). The data or evaluated condition (alert) may be transmitted across a network in S4. A server may receive the data in S5 for processing. The server may save the data in S6. The data may then be displayed in a substantially raw form (for example, as a map of resources across the job site) in S7. Alternatively, the data may be processed in a data analytics step in S8. The analyzed data may then be displayed on an application (for example, as a tracking of resources over time and anticipated completion schedule).

### System: Applications

A variety of software applications may be enabled by use of the device, system, and method disclosed herein. The sensor information and other data provided into the system may be used an accessed to enable certain software, in various examples of embodiments. The system may include an open API. The system may also be accessed through a web-based app portal connected to a cloud server. The open API may allow for programs for control and collection of diagnostic information from active devices or historic data. These programs may assist in automation or augmentation of existing or new organization field operation protocols and tasks.

The system may allow for software for efficient production, including a help plan program. Predictive analytics may also be provided. The system may allow for the ability to subdivide work by prescribed task in discipline trades or expertise. In addition, the system may allow for software for accurate and reliable bidding from the use of the system data. The system may allow for the correct scheduling of the right person, material, etc, during all different phases of construction using prediction analytics. Progress reports, completion dates, individualized employee feedback, whether corrections are followed, potential earnings in each section or phase of building, and diagnostics, may all be enabled using the system and method herein. Security firewalls may be provided to protect the data.

Daily schedules of one or more project workers may also be provided. This data may allow for scheduling functionality. A real-time production model may likewise be able to be generated using the data, system, and method disclosed herein. Detailed and accurate reporting may be produced regarding employees, employee productivity, etc.

The device, system, and method herein may allow for a matrix of regressive data and real-time data which may be generated throughout the organizational use. The data may be used in connection with construction management data-analytic software otherwise known as Enterprise Resource Planning (ERP) software and a customer's Engineer Procurement Construction (EPC) contractors.

The suspension and/or body harness device may further comprise a voice-activating non-invasive user interface that also encapsulates a state of the art MEMS that will autonomously via software provide the vital-production data-package into ERP operating systems (and BIM operating systems) designed to automate or augment job-site management protocols and tasks. In various embodiments, the systems may be one or more cloud mobile collaboration systems. The device, system, and method may allow for track-training of physically demanding, intense cognitive focus, and related behaviors. That every manufacturing/heavy-construction field-employee demonstrates in the process of meeting the physical & cognitive demanding prescribed task objections. The device, system, and method may aid in overcoming obstacles that weather and environmental hazards lay-out along the way of completing their task objections. These advantages may exist throughout all the phases of a construction project. The data produced by the device, system, and method herein may link directly into cloud mobile construction management ERP software-operating-systems to provide advantages to construction management ERP software customers (i.e. design/build-EPC-contractors). The advantages may include increasing the capacity to complete contracts at a faster rate and generate revenue exceeding current or projected profit margins. In addition, the system may provide a mechanism for immediately identifying losses and preventing future losses.

The system may be deployed on suitable servers to enable application across a variety of implementation sites. The data may be used to generate certain predictive analytics and artificial intelligence properties (for example, as applied across bidding for new construction, updating timetables, etc.).

A variety of worker health data (biometric health data) may be provided into the system for evaluation. This data may include Age, body size, fitness level, average breathing rate, average respiration rate, average heart rate, any pre-existing heart conditions, whether their prescribed task is performed while standing or sitting, any current medications, mental health conditions, and average body temperature. The device may evaluate, for example, heart rate and breathing rate during use. This may allow for the system to identify or anticipate possible health issues occurring on the jobsite. The device (suspension, body harness, gloves, earbuds (earplugs)) embedded system may record and track changes in environment, as well as a wearer's biometrics. This information may be provided over a network. In various embodiments, the information may be provided via. 3-D/4-D GPU a real-time data feed from each device micro-environments through-out the various phases of the construction project eco-system. This real-time data feed may be provided to a server for computing algorithms to render assimilated data for services to various software programs. Use of an open API with the system herein may allow heavy industry enterprises to create their own software to benefit their particular needs and turn on or off certain functionality or sensory modes.

The system and method herein may further comprise a field employee intelligent identification. When an organization begins working with an individual, an identification file may be created for use with the system and method herein. Identification factors may be based on DMV license plates. In addition, information regarding one or more of the following non-limiting factors may be used: skill-craft areas of expertise in which a worker specializes in performing, average performance statistics across skilled-craft areas of expertise, training certifications (current), safety certifications (current), accidents reports (any & all), bio-metrics health-vitals that effect heart-rate and breathing-rate (age, body size, fitness level, average breathing and respiration rate, average heart rate, any heart conditions, whether the prescribed task is performed standing or sitting, medications, emotional health, average body temperature), information on all projects worked to date, and attendance score based on timely attendance for those projects and current project.

Additional software may enhance the devices, systems, and methods disclosed herein. For example, a two-way language translator may be used with the devices in order to facilitate communication across the workforce between persons of varying backgrounds. The need for such translation may be automatically detected by way of the embedded microphone and speakers in connection with the servers. Alternatively, translation may be manually enabled when the device is assigned to a worker.

In another example, a time card punch-in software may be used in connection with a position locator for Hot Mapping, Daily Reporting, and Virtual HR program purposes. In other words, the time the employee works may be tracked. In alternative embodiments, a smart identification card having a tag may be used for time tracking in connection with the system disclosed herein. The time performing a prescribed task may be integrated with the production and health data to augment daily reporting of time spent, work completed, and by which skilled trade or sub-contractor.

In a further example, a “create crew” program may be made. A Create Crew program using the device, system, and method herein may allow a field-foreman (or other suitable supervisory employee) through the application dashboard to choose to create a crew of any size. The crew may be provided or selected for a specific craft, task, or objective, in various embodiments. Through the dashboard, which may have one touch crew creator functionality, a field foreman may select the type of work (in relation to the organizational needs) that needs to be performed as well as various tasks associated with that type work application. This application may be preloaded with employee data using a Virtual HR system generated by the system and method herein. The Virtual HR system may autonomously load each of the team members field-employee Intelligent Identification (INID) file. The assimilated data matrix (sensor data with static data) may provide the ability for one-touch computation for an instant crew creator which delivers the most sustainable field crews possible to perform the set task within the various sub-sections of heavy-industry production stage, according to various embodiments.

The virtual HR system and the INID file may be generated using the HR system and employee data described herein.

A sustainable crew generator program may be a further example of a software using the device, system, and method herein. A Sustainable Crew Generator program may create sustainable crews based on deep learning of individual skilled-craft rates of production of each worker. This functionality may be accomplished by coupling together higher and lower field employee's production averages to create a sustainable crew to perform the prescribed task. An example algorithm may be seen below: A field-employee with a high average A+employee with lower average D, {A+D=B} rate of sustainable production flow. The data used may be based in part on averages from pre-employment (when plausible) fitness & mental aptitude exams. These averages may be applied to current pre-established rates of production that are relevant to the cognitive demands & physical energy of movement needed to accomplish the prescribed task being performed.

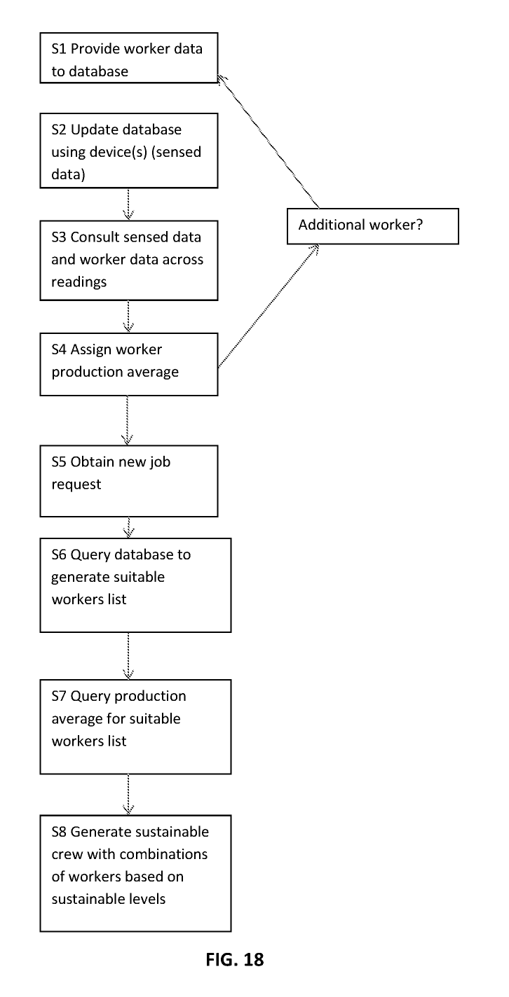

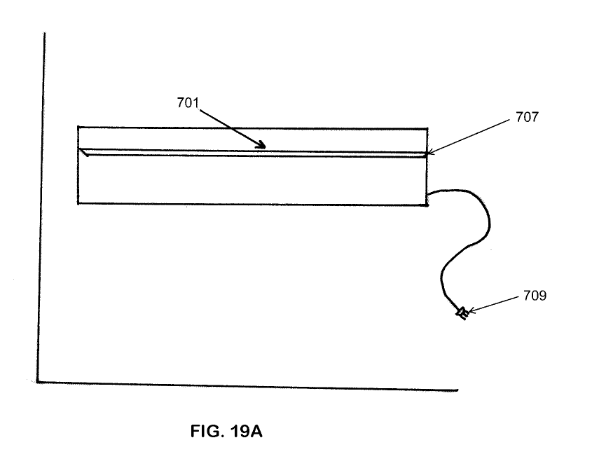

FIG. 18 may provide an example embodiment of a workflow for a sustainable crew generator program. Worker data may be provided into a database in S1 and S2, this data may be consulted or analyzed in S3, and a worker production average may be assigned in S4. When a new job request is obtained or otherwise a new crew is be requested to be generated (S5), the system may query the database to generate a list of skilled workers. The system may then obtain production averages across that set of workers. Then, a sustainable crew may be generated based on those production averages.

The sustainable crew may evaluate productivity ratings over a period of time, for example, over a week. These productivity measurements may be averaged. The productivity values may be a measurement based on mental or physical effort during that time period. The productivity measurement may be related to information acquired upon hiring. The productivity software may also use age, weight, BMI, or other features as one or more factors. The sustainable crew may include pairing higher productivity workers with lower productivity workers in order to result in a standardized efficiency or productivity rate for that crew. The sustainable crew generator may be compared with the requirements of the task at hand. For example, if the task to be performed by the crew entails high mental or physical demand, crew members capable of handling the high mental or physical demand should be used. In contrast, high skilled workers should not be used where there is a low mental or physical demand. In this way, the sustainable crew generator may allow for increased efficiency in use of workers and faster build time.

Currently, a person putting together a crew for construction of a job site may call up a union to find someone to fulfill a role in construction. The disclosed system may allow for building a more comprehensive database of workers. This may advantageously allow for better insurance rates for workers on the jobsite.

A “hot mapping” location sharing program may be a further example of a software using the device, system, and method herein. The Hot Mapping program may enable organization's field-management using the device, system, and method herein to find and track an individual or groups of active field-employees on a 3-D/4-D graphic heat map. This program may record logistic data (e.g. metadata) of field-employees in active work zones and dwell times of active devices may be updated in real-time.

A Safe-Flow program may also be created using the device, system, and method herein. Geo-referencing augmented by the disclosed device, system, and method may help manage dynamic heavy equipment and foot traffic logistics. (e.g. Congestion in hazard safety & or security consciences environments.) The Safe-Flow program application may mitigate and manage dynamic-traffic in congested work-zones by using Hot Mapping location sharing to alert Field-management via. smart phone or tablet SMS link to the application dashboard of 4-D GPU model, in various embodiments. This program may showcase one or more active devices displaying red, moving in and through-out the controlled environment in real-time. In addition, a one touch link functionality may allow for an instant call to involved devices, for example, those having 2-way radios. Software protocols may autonomously archive movement and other relevant data of all devices.

A Safe Watch program may be provided using the device, system and method herein including predictive analytics from compiled blocks of field data generated from the network of active devices to mitigate potential accidents from happening. When an alarming sharp change occurs (e.g. identified alert) with-in an individual Field-employee (key) environmental or bio-metrics while performing a prescribed task with-in the various phases of the construction project, an alert may be sent autonomously to any nearby devices. This functionality may be performed using an embedded digital 2-way radio emergency alarm signal (e.g., provided on the device (suspension, body harness)). The system may simultaneously notify Field management via. Smart phone or tablet SMS. The alert, in various embodiments, may include a link to the Intelligent Work software 3-D/4-D GPU interactive interface were active Field-employees will have a color allotted to them in correspondence to their safe biometric condition & tagged with real-time proficient work levels. ex. Red, Blue, & Green. This functionality may be enabled by the Intelligent Interior Suspension Communication System (or Intelligent Personal Protective Equipment Communication System) or other project job-site staff on their mobile devices. The Intelligent Personal Protective Equipment should be understood to encompass but not be limited to the devices disclosed herein.

A Tool Keep program may be provided using the device, system, and method herein. The program may include chip tracking (or tagging) for monitoring tools & location on job-sites, this allows instant notification of the location of any tool with a RFID chip (QR code, flag, etc.) if an active field employee is using the tool he may be notified in various embodiments via a message. For example, notification may be made using a digital 2-way radio bone-conduction speakers/microphone located within a device including a suspension 100 or application 513 (e.g. application dashboard).

An Intelligent Source Material Profiling System may be provided using the device, system, and method herein. This program protocol may generate a secure electronic virtual shipping document for the organization project management records. Likewise, a Secure Electronic Virtual Shipping Documentation may be provided. This may track shipped orders and provide real-time travel logistics to and through-out the entire building process. The list shown may include but not be limited to: Material(s), Manufacturer, Order date(s), Shipping date(s), Travel method(s) (Including any heavy machinery needed to move the materials and Manufacture specifications on moving the materials). In addition, an intelligent UPC coding program using the device, system, and method herein may be provided. While a UPC code (barcode) is discussed, other forms of tagging such as those disclosed herein including RFID, QR, and the like are likewise in the scope of this disclosure. Like a car license plate the Intelligent UPC-code may be read autonomously and information pertaining to the source material may be archived. List items shown may include but not be limited to: Type(s) of material(s), Count per-pack/bundle, Real-time Location, Ambient temperature finish temperature, Humidity, Manufacture, Distributer, and Order-date(s). In various embodiments, all data may be archived using the system disclosed herein. The code may be applied to both static (construction equipment—light and heavy) and dynamic (people and building materials (i.e. lumber and nails)) materials.

A program for Intelligent Source Material Logistics Management using the device, system, and method herein may likewise be provided. The Intelligent Source Material Management program according to various examples of embodiments digitally connects sub-contractors supplies chains with construction project schedules and deadlines linked via. Project management program portal and an Intelligent UPC-coding (tagging) provided (e.g. fastened, embroidered) on (bulk) dynamic source-materials directly from the manufacture for logistics profiling and management system. At the arrival of job sites materials may be scanned in via. Intelligent UPC-coding (or other tagging 601 mechanism and then scanned again when the material arrives at its designated destination. At the executed time of installation, the organization field-employees device may autonomously scan the barcodes of all bulk source materials used, at the beginning of their interaction, during application process and after-wards as an organization-wide tool a real-time QI (quality improvement) inspector. As an example, this feature may be used in challenging or foreboding environments were highly regulated skilled detailed work application may be carried out. In various embodiments, this data package as well as the correlated data from the device worn by the active field-employee(s) handling and processing the source materials (via field employee intelligent identification system)) may be combined to make an accurate and concise daily/weekly Job-site & incident report. This functionality may be enabled via a Virtual Superintendent program.

In addition, this functionality may be facilitated through an assimilated matrix of data which can help Project Managers in the Virtual Project Manager program to pre-order materials or simply standby incase anything is missed. With self-learning capabilities, virtual Project Manager may perform as an AI (artificial intelligence) assistant. A data matrix combining bulk source material logistics and a Field-employee's production data package may allow for predictive analytics algorithms to place orders of source-materials in precise quantity. A bulk source-material logistics tracking may notify all proper organization Field-management before work can be held up on project schedules. The program may provide for bulk tracking of sub-contractor's source materials through-out production job-sites. This functionality may be enabled via an IR scanner or camera which may, in various embodiments, be located within the embedded digital camera of the device.

A Daily Field Reporting deep learning program may be provided using the device, system, and method herein. The program may augment the daily and weekly reporting protocols for heavy industry projects management. This functionality may be performed by monitoring individual field employee's rates of production bio-metrics and environmental metrics while performing a prescribed task within the various construction/heavy-industry building and/or production phases. A skilled-trade Field-employee's production data-sets may be applied to other relevant data-sets to create a real-time virtual 3-D/4-D graphic model of field production. A data matrix consisting of Skilled-trade Field-employee's production data-sets may be used to quantify values to the answers to the following non-limiting examples of questions: Type of Work being performed? e.g. Concrete, Interior-finishes; Type of Heavy-equipment? e.g. Back-hoe; Time in use? e.g. 2 hrs.; Work was performed for? e.g. Sub-contractor(s); What kind and/or type of source-materials? e.g. Concrete mix, Drywall ⅝″; How much was used? (Bulk) e.g. Sq. yards of concrete/slump, Drywall—3 out of 4 Bunks); Which organizational skilled-trades were involved? e.g. Carpenters, Laborer's; Which sub-contractors were involved? e.g. Electricians, Plumber; Was the prescribed work completed? e.g. yes/no, comments and concerns; If Yes, Was the completed (finish) work done at a quality level? e.g. 1-100 rating scale.

A virtual HR program may likewise be provided using the device, system, and method herein. The Virtual HR may comprise a virtual tool that utilizes assimilated data through-out the system, software, and communication system augmented by the disclosed device, system, and method, for: Vendor billing, Field-employee Payroll, and New Hire Intake.

A virtual project manager program may likewise be provided using the device, system, and method herein. The Augmented Project-management may comprise a virtual tool that utilizes assimilated data through-out the system software and communication system augmented by the device, system, and method herein. The Virtual Project Manager program may automate source material ordering protocols or simply standby incase anything is missed. With self-learning capabilities, virtual Project Manager may perform as an AI assistant.

An intelligent link software may likewise be provided using the device, system, and method herein. The intelligent link software may be used to determine the most cost-effective retailer in relation to project location, transportation, & environmental (weather) logistic. The software may include integration of new-hire Field-employee Intelligent Identification data package as well as Intelligent Source-Materials Logistics data package. These items may augment the building project resource management protocols for both static construction equipment—light and heavy, and dynamic, people and building materials (for example, lumber and nails).

A virtual superintendent software may likewise be provided using the device, system, and method herein. The Virtual Superintendent may comprise a virtual tool that augments standard superintendent protocols by utilizing assimilated data through-out the system software, and communication system augmented by the device, system, and method herein for weekly project reporting protocols.

A virtual foreman software may likewise be provided using the device, system, and method herein. This program may provide daily field-reporting process augmented by the device, system, and method herein. This program may delivers virtual tools to aid and assist organization field supervisors. The virtual foreman may afford the active field-management the ability to be in any and all places at once. The virtual foreman may provide real-time monitoring of field employee's safety and production levels. This functionality may be facilitated using the Hot Mapping application link.

In various embodiments, the system and method herein may provide for topographic mapping surveys.

### System: Charging Device

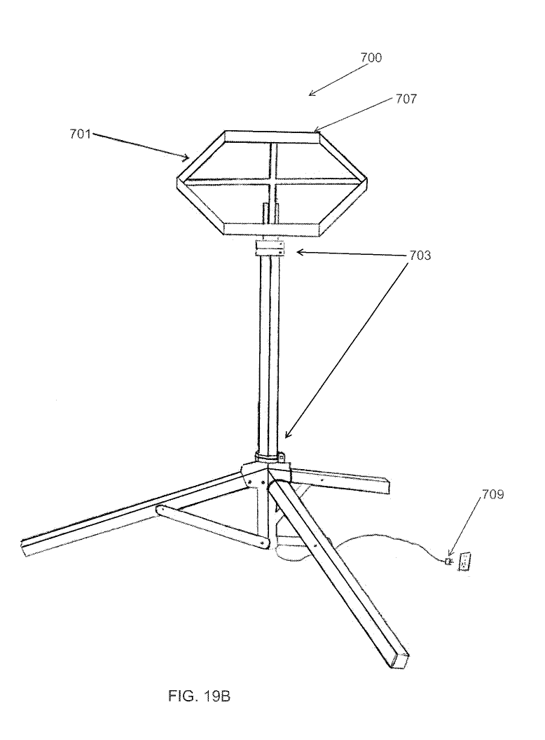

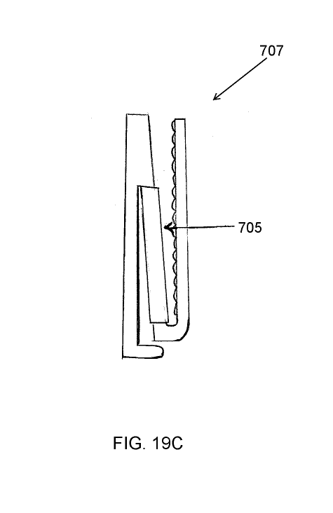

The device, system, and method herein may likewise include a mechanism for charging the devices. FIGS. 19A and 19B show charging devices 700 for use with the disclosed devices (suspension 100, harness 200, gloves 300, earbuds 400) according to various examples of embodiments. The charging device 700 may be understood to include one or more inductive coils. In FIG. 19A, the charging device may comprise an inductive charging metal band 701 be hung on a wall and comprise a plurality of hooks 707 or inductive hooking surface. The charging device 700 may have a plug 709 for accessing a power supply. The charging device 700 may allow for contact or sufficient proximity between the suspension 100 or other device (harness 200, gloves 300, earbuds 400) and charging coils provided within or on an inductive metal charging band 701 or inductive charging metal plate 705. In FIG. 19B, the charging device 700 may have a telescoping tree-like form including telescoping lock nuts 703. The charging device 700 may enable easy storage of the suspensions while facilitating charging by providing for the coils or a charging component in hooks provided in spaced relation. The charging device 700 may allow for convenient storage particularly in the context of trailers which are presently used by many construction companies for daily worker entry and exit (e.g. punching in and out). These trailers may have limited space and the disclosed rack may allow for ease of access when entering and leaving the site. While one substantially linear rack having a plurality of hooks may be shown in FIG. 19A, other shapes, numbers of hooks, and numbers of racks may be contemplated as within the scope of this disclosure.

The charging device may also be provided in the form of a mat (not shown). In this embodiment, the suspensions may connect to the charging device in a substantially mat-like form.

In various embodiments, the charging device 700 may be in the form of a telescoping stand, for example, as shown in FIG. 19B. The telescoping stand may have the format of a tripod and include telescoping lock nuts 703. The charging stand may have a number of arms for hooking devices to be charged onto the stand. The stand may be provided with wireless charging elements for facilitating wireless charging. The stand may be placed on a table or floor for easy placement of the devices.

The charging device may comprise a number of specialized hooks 707, for example, as shown in FIG. 19C. The hooks 707 may be sized to accept any of the devices disclosed herein. For example, the hooks 707 may be sized to accept a body harness 200, suspension 100, gloves 300, and/or earbuds 400.

The devices, system, and method herein provide a number of advantages, according to various examples of embodiments. For example, use of the disclosed items may allow for a heavy-industry organization the ability through verifiable quantified data-sets to show the individual field workers ability to perform his or her set duties in a safe & productive manner. In addition, an insurance adjudicator may be able to access the data to formulate new risk profiles for corporate entities who utilize the disclosed device, system, and method thereby possibly lowering insurance premiums paid by the organization for active employees. Use of a number of the software applications enabled by the device, system, and method herein may generate sustainable work crews that create a safe and more efficient work flow processes. In various embodiments, the sustainable crew may help create a sustainable constructive production working place and a safer working environment by mitigating plausible accidents from cognitive and physical exhaustion.

The disclosed devices (including suspension 100, body harness 200, gloves 300, earbuds 400 etc.) may provide for a safe and comfortable mechanism for providing streamlined and minimal interference while the wearer performs their prescribed tasks and/or duties. The wearable devices may facilitate, in various embodiments, a cloud or satellite. IoT (Internet of Things) mobile interface for Enterprise-Resource-Planning (ERP) collaboration platforms. In various embodiments, the ERP collaboration platform may be a cloud mobile collaboration platform. The devices may be designed in-line for a non-invasive way to monitor real-time individual safety protocols, levels of production, and work-flows proficiencies of heavy construction active field-employees. The data package and programs may automate and/or augment job-site management protocols and tasks. Use of these tools which may be provided or enabled by the device, system, and method herein may assist in meeting and exceeding heavy enterprise organizational needs in levels of production & in safety standards. Use of the device, system, and method herein may allow for improvement over current field-management protocols. In addition, the disclosed device, system, and method may be engineered with an easy access open API for software developers to create deep learning programs.

Various additional components may be contemplated as within the scope of this disclosure. For example, suspensions, harnesses, gloves, earbuds, and nano devices, may all be provided. The system may allow for infrared signals to be sent using a wireless transmitting nanodevice inside of a device. In various embodiments, the devices may be designed for the safe keeping of wireless transmitting nano devices (including high definition nano video). The devices may likewise include an infrared signaler or other transmitting device. The transmitting device may send out signals to devices, manage signal transfers from device(s), receive data from multiple nodes throughout a project, subdivide work by prescribed task in a discipline, trade, or expertise. The system may also comprise a tripod, which may hold infrared signals secure on rough terrain. A lifi receiver may be likewise included for devices streaming data. The system may also include a fence post or stud clamp for fastening an infrared signaler which may include or comprise a tag 601 to these features (e.g. metal posts or studs). These may turn on automatically when a suspension 100, harness 200, glove 300, earbud 400, or other device enters a prescribed area to send and receive data. The receiver may provide external temperatures or readings from the suspension and/or harness, or other device disclosed herein. Predictive analytics may be used based on environmental or other sensor readings (e.g. humidity) to determine what task should be performed next.

The devices disclosed herein may allow for protection of workers. In particular, devices such as the suspension 100, harness 200, earbuds 400, gloves 300, etc. may provide for protection of a worker's head and body from blunt force trauma, excessive noise, etc. The devices may be provided with a display, for example, an OLED display, in various embodiments of the invention. A wireless HDMI receiver may also be provided.

A non-limiting example implementation specification of a suspension 100 and system according to various embodiments may be seen below (while specifics are disclosed, equivalents may be understood within the scope of this disclosure):

Sensor fusion may provide absolute position tracking, all without external transmitters or transceivers
PMF-MVP may be certified for potentially explosive environments in oil & gas, chemical, mining, energy, utilities, aviation
Multiple digital microphones & advanced active noise cancellation: Integrated 91 dB speaker & 3.5 mm audio jack for use with hearing protection.
Local speech recognition in the loudest industrial environments=No-buttons, swiping, or tiring-gestures completely hands-free.

### Self-contained 2 GB RAM/16 GB Flash

Wi-Fi+Bluetooth LE, +MiFi's, & or LiFi or plug-in USB dongles for LTE (Long-Term Evolution (mobile communications standard)). Seamless 4.0 NFC connection tethers from any smart-phone or tablet to the suspension embedded system.
Less than 224 g/8. oz.
The on-board 3300 mAh Lithium Ion battery runs for 12+ hours in real world conditions (Rechargeable, swappable 12+ hour battery)
Miniature atom-based inertial-sensors for extended-operation wireless timing inertial measurement unit (WTIMU). Has an integrated 3-axis gyroscope, a 3-axis accelerometer, & a 3-axis magnetometer. Add micro-technology for positioning, navigation, & timing (Micro-PNT). Together with the integrated highly accurate master timing clock, may simultaneously measure the tracked movement and combine that with timing from the synchronized clock.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

It should be noted that references to relative positions (e.g., “top” and “bottom”) in this description are merely used to identify various elements as are oriented in the Figures. It should be recognized that the orientation of particular components may vary greatly depending on the application in which they are used.

For the purpose of this disclosure, the term “coupled” means the joining of two members directly or indirectly to one another. Such joining may be stationary in nature or moveable in nature. Such joining may be achieved with the two members or the two members and any additional intermediate members being integrally formed as a single unitary body with one another or with the two members or the two members and any additional intermediate members being attached to one another. Such joining may be permanent in nature or may be removable or releasable in nature.

It is also important to note that the construction and arrangement of the system, methods, and devices as shown in the various examples of embodiments is illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements show as multiple parts may be integrally formed, the operation of the interfaces may be reversed or otherwise varied, the length or width of the structures and/or members or connector or other elements of the system may be varied, the nature or number of adjustment positions provided between the elements may be varied (e.g. by variations in the number of engagement slots or size of the engagement slots or type of engagement). The order or sequence of any process or method steps may be varied or re-sequenced according to alternative embodiments. Other substitutions, modifications, changes and omissions may be made in the design, operating conditions and arrangement of the various examples of embodiments without departing from the spirit or scope of the present inventions.

While this invention has been described in conjunction with the examples of embodiments outlined above, various alternatives, modifications, variations, improvements and/or substantial equivalents, whether known or that are or may be presently foreseen, may become apparent to those having at least ordinary skill in the art. Accordingly, the examples of embodiments of the invention, as set forth above, are intended to be illustrative, not limiting. Various changes may be made without departing from the spirit or scope of the invention. Therefore, the invention is intended to embrace all known or earlier developed alternatives, modifications, variations, improvements and/or substantial equivalents.

The technical effects and technical problems in the specification are exemplary and are not limiting. It should be noted that the embodiments described in the specification may have other technical effects and can solve other technical problems.

Aspects of the method described herein are implemented on a software system running on a computer system. To this end, the methods and system may be implemented in, or in association with, a general-purpose software package or a specific purpose software package. As a specific, non-limiting example, the device could be a suspension, body harness, earbud (ear plug), or glove in communication with a cloud storage database and/or mobile device.

The software system described herein may include a mixture of different source codes. The system or method herein may be operated by computer-executable instructions, such as but not limited to, program modules, executable on a computer. Examples of program modules include, but are not limited to, routines, programs, objects, components, data structures, and the like which perform particular tasks or implement particular instructions. The software system may also be operable for supporting the transfer of information within a network.

While the descriptions may include specific devices or computers, it should be understood the system and/or method may be implemented by any suitable device (or devices) having suitable computational means. This may include programmable special purpose computers or general-purpose computers that execute the system according to the relevant instructions. The computer system or portable electronic device can be an embedded system, a personal computer, notebook computer, server computer, mainframe, networked computer, workstation, handheld computer, as well as now known or future developed mobile devices, such as for example, a personal digital assistant, cell phone, smartphone, tablet computer, mobile scanning device, and the like. Other computer system configurations are also contemplated for use with the communication system including, but not limited to, multiprocessor systems, microprocessor-based or programmable electronics, network personal computers, minicomputers, smart watches, and the like. Preferably, the computing system chosen includes a processor suitable for efficient operation of one or more of the various systems or functions or attributes of the communication system described.

The system or portions thereof may also be linked to a distributed computing environment, where tasks are performed by remote processing devices that are linked through a communication network(s). To this end, the system may be configured or linked to multiple computers in a network including, but not limited to, a local area network, wide area network, wireless network, and the Internet. Therefore, information, content, and data may be transferred within the network or system by wireless means, by hardwire connection, or combinations thereof. Accordingly, the devices described herein communicate according to now known or future developed pathways including, but not limited to, wired, wireless, and fiber-optic channels.

In one or more examples of embodiments, data may be stored remotely (and retrieved by the application) or may be stored locally on the user's device in a suitable storage medium. Data storage may be in volatile or non-volatile memory. Data may be stored in appropriate computer-readable medium including read-only memory, random-access memory, CD-ROM, CD-R, CD-RW, magnetic tapes, flash drives, as well as other optical data storage devices. Data may be stored and transmitted by and within the system in any suitable form. Any source code or other language suitable for accomplishing the desired functions described herein may be acceptable for use.

Furthermore, the computer or computers or portable electronic devices may be operatively or functionally connected to one or more mass storage devices, such as but not limited to, a hosted database or cloud-based storage.

The system may also include computer-readable media which may include any computer-readable media or medium that may be used to carry or store desired program code that may be accessed by a computer. The invention can also be embodied as computer-readable code on a computer-readable medium. To this end, the computer-readable medium may be any data storage device that can store data. The computer-readable medium can also be distributed over a network-coupled computer system so that the computer-readable code is stored and executed in a distributed fashion.

1. A head-protection safety apparatus comprising:
a suspension;
a plurality of sensors provided within the suspension;
a wireless data communication device;
a wireless audio communication device; and
a self-illuminating light-emitting diode.

2. A method for communicating information relating to a construction site, the method comprising:
establishing a radio transceiver connection
obtaining a plurality of sensor data from a sensor unit provided on a suspension;
transmitting the sensor data to a server over a local wireless network;
storing the sensor in a database; and
accessing the sensor data through a software.

3. A charging device comprising: a linear rack having a plurality of hooks in communication with a charging coil.

4. A safety monitoring system comprising:
a suspension having one or more sensors, one or more wireless communication devices, and one or more self-illuminating light-emitting diodes;
one or more identification tags;
a server; and
a local area network in communication with the suspension and server.

5. The safety monitoring system of claim 6, further comprising a body harness having one or more sensors in communication with the suspension.

6. The safety monitoring system of claim 6, further comprising one or more gloves having one or more sensors in communication with the suspension.

7. The safety monitoring system of claim 6, further comprising one or more earbuds having one or more sensors in communication with the suspension.

8. The safety monitoring system of claim 6, further comprising a human resources portal.

9. The safety monitoring system of claim 10, further comprising a sustainable crew generator software.
